(19) United States
(12) Patent Application Publication
(10) Pub. No.: US 2015/0329603 A1
(43) Pub. Date: Nov. 19, 2015

(54) Compositions and Methods Related to Parasites

(71) Applicant: CALIFORNIA INSTITUTE OF TECHNOLOGY, PASADENA, CA (US)

(72) Inventors: Erich M. Schwarz, Ithaca, NY (US); Yan Hu, San Diego, CA (US); Igor Antoshechkin, La Crescenta, CA (US); Paul W. Sternberg, Pasadena, CA (US); Raffi V. Aroian, San Diego, CA (US)

(21) Appl. No.: 14/711,386

(22) Filed: May 13, 2015

Related U.S. Application Data
(60) Provisional application No. 61/992,455, filed on May 13, 2014.
(60) Provisional application No. 61/992,481, filed on May 13, 2014.
(60) Provisional application No. 61/992,639, filed on May 13, 2014.
(60) Provisional application No. 61/992,650, filed on May 13, 2014.

(51) Int. Cl. C07K 14/435

(52) U.S. Cl. CPC C07K 14/4354; A61K 39/00

(57) ABSTRACT

In some aspects, the invention relates to compositions and methods for preventing or treating a hookworm infection. In some aspects, the invention relates to nucleic acids, peptides, proteins, antigens, and cells that encode, comprise, and/or express one or more hookworm amino acid sequences, e.g., for use in manufacturing a vaccine.

### PRIORITY CLAIM

This application claims priority to U.S. Provisional Patent Application No. 61/992,455, filed on May 13, 2014, U.S. Provisional Patent Application No. 61/992,481, filed on May 13, 2014, U.S. Provisional Patent Application No. 61/992,639, filed on May 13, 2014, and U.S. Provisional Patent Application No. 61/992,650, filed on May 13, 2014, each of which is hereby incorporated by reference in its entirety.

### GOVERNMENT INTEREST

This invention was made with government support under R01 GM084389 & R01 AI056189 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 8, 2015, is named CTH-01701_SL.txt and is 732,956 bytes in size.

### BACKGROUND

Hookworms (Ancylostoma duodenale, Necator americanus, and Ancylostoma ceylanicum) infect one-tenth of the human race, causing chronic debility. The drugs currently used against hookworms are only partially effective, making new drugs highly desirable. Although effective vaccines against hookworms would be an even better way to lower their abundance, there currently exist no such vaccines.

### SUMMARY

In some aspects, the invention relates to a nucleic acid comprising a nucleotide sequence encoding an amino acid sequence comprising at least 10 consecutive amino acids encoded by an open reading frame in any one of SEQ ID NOS:1-540, and a promoter operably linked to the nucleotide sequence, wherein the promoter is not a hookworm promoter. The nucleotide sequence may encode, for example, the amino acid sequence encoded by any one of SEQ ID NOS:1-540, or the nucleotide sequence may encode an amino acid sequence with at least 95% sequence homology with the amino acid sequence encoded by any one of SEQ ID NOS:1-540.

In some aspects, the invention relates to a method of transforming or transfecting a cell with a nucleic acid described herein.

In some aspects, the invention relates to a cell comprising a nucleic acid described herein.

In some aspects, the invention relates to a method for producing an antigen, comprising incubating a cell as described herein under conditions sufficient to express a nucleotide sequence as described herein, thereby producing the antigen.

In some aspects, the invention relates to a method for preventing or treating a hookworm infection in a subject, comprising administering to the subject a composition comprising an antigen, wherein the antigen comprises an amino acid sequence comprising at least 10 consecutive amino acids encoded by an open reading frame in any one of SEQ ID NOS:1-540. The antigen may comprise, for example, the amino acid sequence encoded by any one of SEQ ID NOS:1-540, or the antigen may comprise an amino acid sequence with at least 95% sequence homology with the amino acid sequence encoded by any one of SEQ ID NOS:1-540.

In some aspects, the invention relates to a method for preventing or treating a hookworm infection in a subject, comprising administering to the subject a composition comprising a nucleic acid, wherein the nucleic acid comprises a nucleotide sequence encoding an amino acid sequence comprising at least 10 consecutive amino acids encoded by an open reading frame in any one of SEQ ID NOS:1-540. The nucleotide sequence may encode, for example, the amino acid sequence encoded by any one of SEQ ID NOS:1-540, or the nucleotide sequence may encode an amino acid sequence with at least 95% sequence homology with the amino acid sequence encoded by any one of SEQ ID NOS:1-540.

In some aspects, the invention relates to a peptide, protein, or antigen comprising an amino acid sequence comprising at least 10 consecutive amino acids encoded by an open reading frame in any one of SEQ ID NOS:1-540. The amino acid sequence may comprise, for example, the amino acid sequence encoded by any one of SEQ ID NOS:1-540, or the amino acid may comprise an amino acid sequence with at least 95% sequence homology with the amino acid sequence encoded by any one of SEQ ID NOS:1-540.

### DESCRIPTION OF THE FIGURES

FIG. 1. Overview of search strategy for identifying drug targets specific to multiple parasites, exemplified with a specific application to the hookworm Ancylostoma ceylanicum.

### DETAILED DESCRIPTION

### Definitions

The articles “a” and “an” are used herein to refer to one or to more than one (i.e., to at least one) of the grammatical object of the article. By way of example, “an element” means one element or more than one element.

Throughout this specification, the word “comprise” or variations such as “comprises” or “comprising” will be understood to imply the inclusion of a stated integer or groups of integers but not the exclusion of any other integer or group of integers.

As used herein, the terms “effective amount” and “therapeutically effective amount” mean a dosage sufficient to produce a desired result, e.g., to prevent or treat a hookworm infection in a subject.

The term “prevent” is art-recognized, and when used in relation to a condition, such as a hookworm infection, is well understood in the art, and includes administration of a composition which reduces the likelihood of, or delays the onset of, the condition in a subject relative to a subject which does not receive the composition. Thus, prevention of hookworm includes, for example, reducing the likelihood that a subject receiving the composition will develop a hookworm infection relative to a subject that does not receive the composition, and/or reducing the severity of a subsequent hookworm infection, on average, in a treated population versus an untreated control population, e.g., by a statistically and/or clinically significant amount.

“SEQ ID NOS:1-540” refers to each of the 540 nucleotide sequences included in the associated Sequence Listing file (i.e., each nucleotide sequence from SEQ ID NO:1 to SEQ ID NO:540). Accordingly, “any one of SEQ ID NOS:1-540” refers to any one of the 540 nucleotide sequences in the associated Sequence Listing file (i.e., any one of the 540 nucleotide sequences from SEQ ID NO:1 to SEQ ID NO:540).

The term “sequence homology” is used interchangeably with “sequence identity” herein. Sequence homology and sequence identity may be calculated using programs such as a Clustal or BLAST. The “tblastn” program, for example, translates an inputted nucleotide sequence in each reading frame to arrive six amino acid sequences, and the program searches nucleotide sequence databases translated in each reading frame to identify nucleotide sequences that encode amino acid sequences with homology to an amino acid sequence encoded by the input nucleotide sequence. Thus, tblastn is particularly useful for identifying a nucleotide sequence encoding an amino acid sequence, which has sequence homology with an amino acid sequence encoded by a different nucleotide sequence. Both Clustal and BLAST may introduce gaps in order to maximize a sequence homology calculation; for calculating sequence homology or sequence identity with the introduction of gaps, default weights may be used for weighting gaps (e.g., gap opening, gap extension, etc.) relative to homology/identity. For each nucleotide sequence, thymine (“T”) is equivalent to uracil (“U”) for calculating sequence homology or sequence identity.

The terms “transforming” and “transfecting” are used interchangeably herein and refer to the introduction of a nucleic acid into a cell, e.g., to produce a recombinant cell. A nucleotide sequence encoded by the nucleic acid may or may not be inheritable to the progeny of the cell. Transfection, for example, may be stable (i.e. the nucleic acid is integrated into the genome of a cell and thereby inheritable to the progeny of the cell) or transient (i.e., wherein the expression of a nucleotide sequence encoded by the nucleic acid is lost after a period of time).

As used herein, the terms “treat”, “treating”, and “treatment” include inhibiting the condition, e.g., reducing the onset or symptoms of a condition, disorder, or disease, such as a hookworm infection. These terms also encompass therapy. Treatment means any manner in which the symptoms of a condition, disorder, or disease are ameliorated or otherwise beneficially altered. Preferably, the subject in need of such treatment is a mammal, such as a human, pet (e.g., cat or dog), or farm animal.

### I. NUCLEIC ACIDS

In some aspects, the invention relates to a nucleic acid comprising a nucleotide sequence encoding an amino acid sequence, e.g., an antigen. An epitope may be, for example, as small as 8 amino acids, and thus, an amino acid sequence as short as 8 amino acids may be sufficient to produce an immune response in a subject. Thus, a nucleic acid may be useful for producing an antigen even if the nucleic acid encodes only a short fragment of a protein, e.g., as few as 5, 6, 7, 8, 9, or 10 amino acids. Additionally, the codons of a nucleotide sequence may be altered, for example, to optimize the expression of an amino acid sequence in a cell or for molecular cloning, such as to introduce or remove restriction sites. Thus, a nucleotide sequence encoding an amino acid sequence for expression in a cell may vary from a nucleotide sequence obtained, for example, by sequencing a genome. Further, an amino acid sequence that varies from a naturally-occurring amino acid sequence (e.g., a hookworm sequence) may nevertheless provoke an immune response against the naturally-occurring sequence. Similarly, an amino acid sequence from one species of hookworm may vary from an orthologous amino acid sequence in a different species of hookworm. Thus, a nucleotide sequence may encode an amino acid sequence that provokes an immune response against a different amino acid sequence (e.g., a hookworm sequence), even though the two sequences vary, so long as the two sequences have sufficient sequence homology (e.g., at least 95% sequence homology).

In some embodiments, the invention relates to a nucleic acid comprising a nucleotide sequence encoding an amino acid sequence comprising at least 10 consecutive amino acids encoded by an open reading frame in any one of SEQ ID NOS:1-540. The nucleotide sequence may encode an amino acid sequence comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 220, 240, 260, 280, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, or 2000 consecutive amino acids encoded by an open reading frame in any one of SEQ ID NOS:1-540. The nucleotide sequence may encode an amino acid sequence with at least about 95% sequence homology with an amino acid sequence comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 220, 240, 260, 280, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, or 2000 consecutive amino acids encoded by an open reading frame in any one of SEQ ID NOS:1-540. The nucleotide sequence may encode an amino acid sequence with at least about 85%, 86%, 87%, 88%, 89%, 90%, 91%, 92%, 93%, 94%, 95%, 96%, 97%, 98%, 99%, 99.5%, 99.8%, 99.9%, or even 100% sequence homology with an amino acid sequence comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 220, 240, 260, 280, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, or 2000 consecutive amino acids encoded by an open reading frame in any one of SEQ ID NOS:1-540.

The nucleotide sequence may encode an amino acid sequence encoded by an open reading frame in any one of SEQ ID NOS:1-540. The nucleotide sequence may encode an amino acid sequence with at least about 95% sequence homology with an amino acid sequence encoded by an open reading frame in any one of SEQ ID NOS:1-540. The nucleotide sequence may encode an amino acid sequence with at least about 85%, 86%, 87%, 88%, 89%, 90%, 91%, 92%, 93%, 94%, 95%, 96%, 97%, 98%, 99%, 99.5%, 99.8%, 99.9%, or even 100% sequence homology with an amino acid sequence encoded by an open reading frame in any one of SEQ ID NOS:1-540.

An open reading frame includes any nucleotide sequence that encodes consecutive amino acids. In preferred embodiments, the open reading frame is Frame 1, read from 5′ to 3′, for SEQ ID NOS:1-532 and SEQ ID NO:537-540, and Frame 3, read from 5′ to 3′, for SEQ ID NO:533-536. In general, however, each sequence of SEQ ID NOS:1-540, comprises an open reading frame that spans the entire length of the nucleotide sequence, terminating in a stop codon (e.g., Frame 1, read from 5′ to 3′), and thus, any nucleotide sequence comprising at least 9 consecutive nucleotides in SEQ ID NOS:1-540 will encode an amino acid sequence (i.e., at least 2 consecutive amino acids) encoded by the preferred open reading frame.

In some preferred embodiments, the nucleic acid comprises a promoter operably linked to the nucleotide sequence encoding the amino acid sequence, i.e., to drive the transcription of the nucleotide sequence in a cell. The nucleic acid may not comprise a promoter, for example, when the nucleic acid is RNA, when the nucleic acid is used in a method to make a cell or nucleic acid (e.g., according to certain embodiments of the invention), or when the nucleic acid is used in a vaccine. In preferred embodiments, the promoter is linked to the nucleotide sequence such that transcripts of the nucleotide sequence may be translated in a preferred open reading frame (Frame 1, read from 5′ to 3′, for SEQ ID NOS:1-532 and SEQ ID NOS:537-540, and Frame 3, read from 5′ to 3′, for SEQ ID NOS:533-536).

In preferred embodiments, the promoter is not a hookworm promoter. In some embodiments, the promoter can drive transcription of the nucleotide sequence in a bacterium, yeast, fungal cell, plant cell, insect cell, or mammalian cell. In preferred embodiments, the promoter can drive transcription of the nucleotide sequence in Escherichia coli, Bacillus subtilis, Pseudomonas fluorescens, Leishmania tarentolae, Saccharomyces cerevisiae, Pichia Pastoris, Nicotiana, Drosophila melanogaster, Spodoptera frugiperda, Trichoplusia ni, Gallus gallus, Mus musculus, Sus scrofa, Ovis aries, Capra aegagrus, Bos taurus, Sf9 cells, Sf21 cells, Schneider 2 cells, Schneider 3 cells, High Five cells, NS0 cells, Chinese Hamster Ovary (“CHO”) cells, Baby Hamster Kidney cells, COS cells, Vero cells, HeLa cells, or HEK 293 cells.

In some preferred embodiments, the promoter can drive transcription of the nucleotide sequence in Escherichia coli, Saccharomyces cerevisiae, or CHO cells.

In some embodiments, the nucleic acid comprises an origin of replication, e.g., for replication in a cloning cell or an expression cell. In some embodiments, the nucleic acid encodes at least one affinity tag, e.g., for purifying an antigen, such as AviTag, Calmodulin-tag, polyglutamate tag, E-tag, FLAG-tag, HA-tag, His-tag, Myc-tag, S-tag, SBP-tag, Softag 1, Softag 3, Strep-tag, TC tag, V5 tag, VSV-tag, Xpress tag, Isopeptag, and/or SpyTag. In some embodiments, the nucleic acid encodes a chaperone, such as glutathione S-transferase, to increase the expression or the stability of an antigen. In some embodiments, the nucleic acid encodes a protease cleavage site, e.g., for removing an affinity tag or chaperone, such as a protease cleavage site for cleavage by enteropeptidase, Factor Xa, rhinovirus 3C protease, TEV protease, or thrombin. In some embodiments, the nucleic acid encodes a methionine, e.g., for removing an affinity tag or chaperone by hydrolysis with cyanogen bromide.

In some embodiments, the nucleic acid is a plasmid or linear nucleic acid.

### II. CELLS COMPRISING A NUCLEOTIDE SEQUENCE

a. Methods for Transforming or Transfecting a Cell

In some aspects, the invention relates to method for transforming or transfecting a cell, comprising transforming or transfecting a cell with a nucleic acid comprising a nucleotide sequence as described herein, supra. For example, the nucleic acid may comprise a nucleotide sequence that encodes an amino acid sequence with at least about 85%, 86%, 87%, 88%, 89%, 90%, 91%, 92%, 93%, 94%, 95%, 96%, 97%, 98%, 99%, 99.5%, 99.8%, 99.9%, or even 100% sequence homology with an amino acid sequence comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 220, 240, 260, 280, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, or 2000 consecutive amino acids encoded by an open reading frame in any one of SEQ ID NOS:1-540. The nucleic acid may or may not comprise a promoter. In some embodiments, the nucleic acid consists of a nucleic acid as described herein, supra.

b. Cells that may be Transformed or Transfected

In some embodiments, the cell is a bacterium, yeast, fungal cell, plant cell, insect cell, or mammalian cell. The cell may be, for example, a cloning cell or an expression cell. Suitable expression cells include Escherichia coli, Bacillus subtilis, Pseudomonas fluorescens, Leishmania tarentolae, Saccharomyces cerevisiae, Pichia Pastoris, Nicotiana, Drosophila melanogaster, Spodoptera frugiperda, Trichoplusia ni, Gallus gallus, Mus musculus, Sus scrofa, Ovis aries, Capra aegagrus, Bos taurus, Sf9 cells, Sf21 cells, Schneider 2 cells, Schneider 3 cells, High Five cells, NS0 cells, Chinese Hamster Ovary (“CHO”) cells, Baby Hamster Kidney cells, COS cells, Vero cells, HeLa cells, and HEK 293 cells. In some preferred embodiments, the cell is an Escherichia coli, Saccharomyces cerevisiae, or CHO cell.

c. Transformed/Transfected Cells

In some aspects, the invention relates to any one of the aforementioned cells comprising a nucleotide sequence as described herein, supra. For example, the nucleotide sequence may encode an amino acid sequence with at least about 85%, 86%, 87%, 88%, 89%, 90%, 91%, 92%, 93%, 94%, 95%, 96%, 97%, 98%, 99%, 99.5%, 99.8%, 99.9%, or even 100% sequence homology with an amino acid sequence comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 220, 240, 260, 280, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, or 2000 consecutive amino acids encoded by an open reading frame in any one of SEQ ID NOS:1-540. In some preferred embodiments, i.e., when the cell is an expression cell, the nucleotide sequence is operably linked to a promoter. The nucleotide sequence may not be operably linked to a promoter, for example, when the cell is a cloning cell.

### III. METHODS FOR PRODUCING AN ANTIGEN

a. Peptides and proteins comprising an antigen

In some aspects, the invention relates to a peptide or protein comprising an antigen. The peptide or protein may consist essentially of the antigen, or the peptide or protein may be, for example, a fusion protein that comprises the antigen. The antigen may comprise an amino acid sequence comprising at least 10 consecutive amino acids encoded by an open reading frame in any one of SEQ ID NOS:1-540. The antigen may comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 220, 240, 260, 280, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, or 2000 consecutive amino acids encoded by an open reading frame in any one of SEQ ID NOS:1-540. The antigen may comprise an amino acid sequence with at least about 95% sequence homology with an amino acid sequence comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 220, 240, 260, 280, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, or 2000 consecutive amino acids encoded by an open reading frame in any one of SEQ ID NOS:1-540. The antigen may comprise an amino acid sequence with at least about 85%, 86%, 87%, 88%, 89%, 90%, 91%, 92%, 93%, 94%, 95%, 96%, 97%, 98%, 99%, 99.5%, 99.8%, 99.9%, or even 100% sequence homology with an amino acid sequence comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 220, 240, 260, 280, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, or 2000 consecutive amino acids encoded by an open reading frame in any one of SEQ ID NOS:1-540.

The antigen may comprise an amino acid sequence encoded by an open reading frame in any one of SEQ ID NOS:1-540. The antigen may comprise an amino acid sequence with at least about 95% sequence homology with an amino acid sequence encoded by an open reading frame in any one of SEQ ID NOS:1-540. The antigen may comprise an amino acid sequence with at least about 85%, 86%, 87%, 88%, 89%, 90%, 91%, 92%, 93%, 94%, 95%, 96%, 97%, 98%, 99%, 99.5%, 99.8%, 99.9%, or even 100% sequence homology with an amino acid sequence encoded by an open reading frame in any one of SEQ ID NOS:1-540.

The peptide or protein may comprise at least one affinity tag, e.g., for purification, such as AviTag, Calmodulin-tag, polyglutamate tag, E-tag, FLAG-tag, HA-tag, His-tag, Myc-tag, S-tag, SBP-tag, Softag 1, Softag 3, Strep-tag, TC tag, V5 tag, VSV-tag, Xpress tag, Isopeptag, and/or SpyTag. The peptide or protein may comprise a chaperone, such as glutathione 5-transferase, e.g., to increase the expression or stability of an antigen. In some embodiments, the peptide or protein comprises a protease cleavage site, e.g., for removing an affinity tag or chaperone, such as a protease cleavage site for cleavage by enteropeptidase, Factor Xa, rhinovirus 3C protease, TEV protease, or thrombin. In some embodiments, the peptide or protein comprises a methionine, e.g., for removing an affinity tag or chaperone by hydrolysis with cyanogen bromide.

b. Methods for producing an antigen

In some aspects, the invention relates to a method for producing a peptide or protein, comprising incubating a cell as described herein, i.e., an expression cell comprising a nucleotide sequence as described herein, under conditions sufficient to express the nucleotide sequence, thereby producing the peptide or protein. The method may further comprise purifying and/or isolating the peptide or protein, e.g., by centrifugation, filtration, an affinity tag, and/or chromatography, such as ion exchange chromatography, size exclusion chromatography, affinity chromatography, etc.

In some aspects, the invention relates to a method for producing an antigen, comprising incubating a cell as described herein, i.e., an expression cell comprising a nucleotide sequence as described herein, under conditions sufficient to express the nucleotide sequence, thereby producing the antigen. The method may further comprise purifying and/or isolating the antigen, e.g., by centrifugation, filtration, an affinity tag, and/or chromatography, such as ion exchange chromatography, size exclusion chromatography, affinity chromatography, etc.

### IV. PEPTIDES, PROTEINS, AND ANTIGENS

In some aspects, the invention relates to a peptide or protein, wherein the peptide or protein comprises at least 10 consecutive amino acids encoded by an open reading frame in any one of SEQ ID NOS:1-540. The peptide or protein may comprise an amino acid sequence comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 220, 240, 260, 280, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, or 2000 consecutive amino acids encoded by an open reading frame in any one of SEQ ID NOS:1-540. The peptide or protein may comprise an amino acid sequence with at least about 95% sequence homology with an amino acid sequence comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 220, 240, 260, 280, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, or 2000 consecutive amino acids encoded by an open reading frame in any one of SEQ ID NOS:1-540. The peptide or protein may comprise an amino acid sequence with at least about 85%, 86%, 87%, 88%, 89%, 90%, 91%, 92%, 93%, 94%, 95%, 96%, 97%, 98%, 99%, 99.5%, 99.8%, 99.9%, or even 100% sequence homology with an amino acid sequence comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 220, 240, 260, 280, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, or 2000 consecutive amino acids encoded by an open reading frame in any one of SEQ ID NOS:1-540.

The peptide or protein may comprise an amino acid sequence encoded by an open reading frame in any one of SEQ ID NOS:1-540. The peptide or protein may comprise an amino acid sequence with at least about 95% sequence homology with an amino acid sequence encoded by an open reading frame in any one of SEQ ID NOS:1-540. The peptide or protein may comprise an amino acid sequence with at least about 85%, 86%, 87%, 88%, 89%, 90%, 91%, 92%, 93%, 94%, 95%, 96%, 97%, 98%, 99%, 99.5%, 99.8%, 99.9%, or even 100% sequence homology with an amino acid sequence encoded by an open reading frame in any one of SEQ ID NOS:1-540.

The peptide or protein may be an antigen, or the peptide or protein may comprise an antigen. In some embodiments, the peptide or protein is not an antigen (and does not comprise an antigen), e.g., wherein the peptide or protein is administered to modulate the immune system of a subject.

### V. PHARMACEUTICAL FORMULATIONS COMPRISING A PEPTIDE, PROTEIN, ANTIGEN, OR NUCLEIC ACID

In some aspects, the invention relates to a composition comprising a peptide, protein, antigen, or nucleic acid as described herein. The composition may be formulated for injection, e.g., the composition may be a liquid. The composition may be formulated for injection into a subject, such as a human subject. The composition may be sterile. The composition may be a pharmaceutical composition, such as a sterile, injectable pharmaceutical composition. The composition may be formulated for intramuscular or subcutaneous injection. In some embodiments, the composition is formulated for transdermal, intradermal, transmucosal, nasal, inhalational, or enteral administration.

The composition may comprise a peptide, protein, antigen, or nucleic acid, as described herein, in a pharmaceutically acceptable carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

Pharmaceutically acceptable diluents include saline and aqueous buffer solutions. Pharmaceutical compositions suitable for injection include sterile aqueous solutions (where water soluble) or dispersions and sterile powders for the extemporaneous preparation of sterile injectable solutions or dispersions. Isotonic agents, for example, sugars, polyalcohols such as mannitol and sorbitol, and/or sodium chloride may be included in the pharmaceutical composition. In all cases, the composition should be sterile and should be fluid. It should be stable under the conditions of manufacture and storage and must include preservatives that prevent contamination with microorganisms, such as bacteria and fungi. Dispersions can also be prepared in glycerol, liquid polyethylene glycols, and mixtures thereof and in oils. Under ordinary conditions of storage and use, these preparations may contain a preservative to prevent the growth of microorganisms.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms in the pharmaceutical composition can be achieved by various antibacterial and antifungal agents, for example, parabens, chlorobutanol, phenol, ascorbic acid, thimerosal, and the like.

Compositions may be formulated in dosage unit form for ease of administration and uniformity of dosage. Dosage unit form refers to physically discrete units suited as unitary dosages for a mammalian subject; each unit contains a predetermined quantity of active material (e.g., the peptide, protein, antigen, or nucleic acid) calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects.

For lung instillation, aerosolized solutions are used. In sprayable aerosol preparations, the active protein may be in combination with a solid or liquid inert carrier material. The compositions may also be packaged in a squeeze bottle or in admixture with a pressurized volatile, normally gaseous propellant. The aerosol preparations can contain solvents, buffers, surfactants, and antioxidants in addition to the protein of the invention.

Other pharmaceutically acceptable carriers for the compositions according to the present invention are liposomes, pharmaceutical compositions in which the active peptide, protein, antigen, or nucleic acid is contained either dispersed or variously present in corpuscles consisting of aqueous concentric layers adherent to lipidic layers. The peptide, protein, antigen, or nucleic acid is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature. Those skilled in the art will appreciate other suitable embodiments of the present liposomal formulations.

### VI. METHODS FOR PREVENTING OR TREATING A HOOKWORM INFECTION

a. Methods comprising administering a peptide, protein, antigen, or nucleic acid

In some aspects, the invention relates to a method for preventing or treating a hookworm infection in a subject, comprising administering to the subject a composition comprising a peptide or protein as described herein. In some embodiments, the invention relates to a method for preventing or treating a hookworm infection in a subject, comprising administering to the subject a composition comprising an antigen as described herein. In some embodiments, the invention relates to a method for preventing or treating a hookworm infection in a subject, comprising administering to the subject a composition comprising a nucleic acid as described herein.

The hookworm infection may be caused, for example, by Ancylostoma duodenale, Necator americanus, or Ancylostoma ceylanicum. The hookworm infection may be caused by Ancylostoma braziliense or Ancylostoma tubaeforme. In some embodiments, the hookworm infection is caused by Ancylostoma caninum.

Administering the composition may comprise any suitable means of delivering a peptide, protein, antigen, or nucleic acid to elicit an immune response. Administering a composition preferably comprises parenteral administration. In preferred embodiments, the composition is administered by subcutaneous or intramuscular injection. Administering a peptide, protein, antigen, or nucleic acid may comprise transdermal, intradermal, transmucosal, nasal, inhalational, or enteral administration.

b. Subjects

The subject may be any organism susceptible to a hookworm infection or any organism that may carry and/or transmit a hookworm. In some embodiments, the subject is selected from murines, felines, canines, ovines, porcines, bovines, equines, and primates. For example, the subject may be selected from Felis catus, Canis lupus familiaris, and Homo sapiens. In some embodiments, the subject is a golden hamster (Mesocricetus auratus). The subject may or may not have a hookworm infection. For example, the subject may have been exposed to a hookworm, the subject may be at risk of hookworm infection, or the subject may be visiting a location associated with an elevated risk of hookworm infection. In some embodiments, the subject does not have a hookworm infection and the subject does not have an elevated risk of hookworm infection.

### VII. METHODS FOR MODULATING AN IMMUNE RESPONSE IN A SUBJECT

a. Methods comprising administering a peptide, protein, antigen, or nucleic acid

In some aspects, the invention relates to a method for modulating an immune response in a subject, comprising administering to the subject a composition comprising a peptide or protein as described herein. In some embodiments, the invention relates to a method for modulating an immune response in a subject, comprising administering to the subject a composition comprising an antigen as described herein. In some embodiments, the invention relates to a method for modulating an immune response in a subject, comprising administering to the subject a composition comprising a nucleic acid as described herein.

In some embodiments, modulating an immune response in a subject relates to increasing an immune response, e.g., against the peptide, protein, antigen, or nucleic acid. For example, administering the composition to a subject may cause the subject to mount an immune response against the peptide, protein, antigen, or nucleic acid.

In other embodiments, modulating an immune response in a subject relates to decreasing an immune response, e.g., an autoimmune response or an immune response associated with a medical treatment, such as a transplant or biologic therapy. For example, certain aspects of the invention relate to the ability of hookworms to dampen the immune systems of their hosts. Hookworm nucleotide sequences encoding proteins that are likely to be immunosuppressive include ASPR genes (SEQ ID NOS:1-187), mammalian-like lectin genes (SEQ ID NOS:188-203), and protease and protease inhibitor genes (SEQ ID NOS:405-540).

Administering the composition may comprise any suitable means for delivering a peptide, protein, antigen, or nucleic acid to a subject. Administering a composition preferably comprises parenteral administration. In some embodiments, the composition is administered by subcutaneous, intramuscular, or intravenous injection. Administering a peptide, protein, antigen, or nucleic acid may comprise transdermal, intradermal, transmucosal, nasal, inhalational, or enteral administration.

b. Subjects

In some embodiments, the subject is selected from murines, felines, canines, ovines, porcines, bovines, equines, and primates. For example, the subject may be selected from Homo sapiens and Mus musculus. The subject may have an autoimmune disease or condition. In some embodiments, the subject is in need of immunosuppression.

### EXEMPLIFICATION

The present description is further illustrated by the following examples, which should not be construed as limiting in any way. The contents of all cited references (including literature references, issued patents, and published patent applications) are hereby expressly incorporated by reference. When definitions of terms in documents that are incorporated by reference herein conflict with those used herein, the definitions used herein govern.

### Example 1

### Identification of the ASPR Protein Family

Example 1 describes a new family of protein-coding genes in Ancylostoma ceylanicum, called ASPRs. They have the following traits, which indicate that their products might be useful vaccines: they are distantly related to Ancylostoma Secreted Proteins (“ASPs”), which are suspected to enable parasitic infection in some manner which may include immunosuppression; like ASPs, they are strongly upregulated at the onset of A. ceylanicum infection in vivo; like ASPs, their gene products are predicted to be secreted; and an ASPR of the parasitic nematode Heligmosomoides polygyrus bakeri has been biochemically shown to be secreted into the host during infection. The predicted coding DNA sequences for A. ceylanicum ASPR genes are disclosed in SEQ ID NO:1 to SEQ ID NO:187, which encode amino acid sequences that may serve as useful antigens for preventing or treating a hookworm infection.

The genome and infectious transcriptome of Ancylostoma ceylanicum, a hookworm which infects both humans and other mammals, and predicted its genome to contain 37,016 protein-coding genes was sequenced. To find which genes were specifically activated during infection, the expression profile was assessed by RNA-seq analysis at the following infection stages (with A. ceylanicum in golden hamsters): infectious third-stage larvae, before infection (L3i); 24 hours after infection in vivo (in the stomach lining of the hamster; 24PI); 24 hours after incubation in hookworm culture medium, a commonly used synthetic model of infection (24HCM); 5 days after infection (5.D); 12 days after infection (12.D); 17 and 19 days after infection (17.D and 19.D). Genes were classified both by known protein motifs (through HMMER 3.0/Pfam-A 26 and InterProScan 4.8) and by uncharacterized protein motifs and homologies (through HMMER 3.0/Pfam-B 26 and OrthoMCL 1.3). For OrthoMCL, the predicted A. ceylanicum protein-coding genes were compared to those of ten other nematodes from WormBase release WS230 (Ascaris suum, Brugia malayi, Bursaphelenchus xylophilus, Caenorhabditis elegans, C. briggsae, Dirofilaria immitis, Haemonchus contortus, Meloidogyne hapla, Pristionchus pacificus, and Trichinella spiralis) and to those of two mammals from Ensembl release 70 (Homo sapiens and Mus musculus). Sources of these proteomes are listed in Table 1.

TABLE 1
Sources of the nematode and mammalian proteomes that were compared in order to define
new orthology groups in A. ceylanicum, or to define the ASPR protein family.

Ancylostoma ceylanicum (zoonotic hookworm parasite): Proprietary; see Sequence Listing
Ancylostoma caninum, translated ESTs (hookworm parasite of dogs): http://nematode.net/Data/
translations_ftp/AC.trans.final.faa.
Ascaris suum (roundworm parasite of pigs, closely related to the human roundworm parasite
Ascaris lumbricoides): ftp://ftp.sanger.ac.uk/pub2/wormbase/releases/WS230/species/a_suum/
a_suum.WS230.protein.fa.gz.
Brugia malayi (parasitic nematode of humans): ftp://ftp.sanger.ac.uk/pub2/wormbase/releases/
WS230/species/b_malayi/b_malayi.WS230.protein.fa.gz.
Bursaphelenchus xylophilus (parasitic nematode of trees): ftp://ftp.sanger.ac.uk/pub2/wormbase/
releases/WS230/species/b_xylophilus/b_xylophilus.WS230.protein.fa.gz.
Caenorhabditis angaria (non-parasitic nematode, closely related to C. elegans): ftp://
ftp.sanger.ac.uk/pub2/wormbase/releases/WS230/species/c_angaria/c_angaria.WS230.protein.fa.gz.
Caenorhabditis brenneri (non-parasitic nematode, closely related to C. elegans): ftp://
ftp.sanger.ac.uk/pub2/wormbase/releases/WS230/species/c_brenneri/c_brenneri.WS230.protein.
fa.gz.
Caenorhabditis briggsae (non-parasitic nematode, closely related to C. elegans): ftp://ftp.sanger.
ac.uk/pub2/wormbase/releases/WS230/species/c_briggsae/c_briggsae.WS230.protein.fa.gz.
Caenorhabditis elegans (experimentally well-characterized non-parasitic nematode): ftp://ftp.
sanger.ac.uk/pub2/wormbase/releases/WS230/species/c_elegans/c_elegans.WS230.protein.fa.gz.
Caenorhabditis remanei (non-parasitic nematode, closely related to C. elegans): ftp://
ftp.sanger.ac.uk/pub2/wormbase/releases/WS230/species/c_remanei/c_remanei.WS230.protein.
fa.gz.
Caenorhabditis japonica (non-parasitic nematode, closely related to C. elegans): ftp://
ftp.sanger.ac.uk/pub2/wormbase/releases/WS230/species/c_japonica/c_japonica.WS230.protein.
fa.gz.
Caenorhabditis sp. 5 (non-parasitic nematode, closely related to C. elegans): ftp://
ftp.sanger.ac.uk/pub2/wormbase/releases/WS230/species/c_sp5/c_sp5.WS230.protein.fa.gz.
Caenorhabditis sp. 11 (non-parasitic nematode, closely related to C. elegans): ftp://
ftp.sanger.ac.uk/pub2/wormbase/releases/WS230/species/c_sp11/c_sp11.WS230.protein.fa.gz.
Cooperia oncophora, translated ESTs (parasitic nematode of sheep and goats): http://
nematode.net/Data/transcript_assembly_ftp/Cooperia_Oncophora.p4ePro.fsa.
Dictyocaulus viviparus, translated ESTs (parasitic nematode of cows): http://nematode.net/Data/
transcript_assembly_ftp/D.viviparus_pro.faa.
Dirofilaria immitis (parasitic nematode of dogs): http://nematodes.org/downloads/
959nematodegenomes/blast/db2/nDi.2.2.2.aug.proteins.fasta.gz, dated 07-Aug-2012 12:26.
Haemonchus contortus (parasitic nematode of sheep, closely related to A. ceylanicum). for
OrthoMCL, two sets of gene predictions were used (from MAKER2 and AUGUSTUS), treated
as two species for the purposes of analysis, from Schwarz et al. Genome Biol., 14(8): R89 (2013).
For psi-BLAST, only the MAKER2 set of predictions was used, but a set of gene predictions
from WormBase WS230 was also used: ftp://ftp.sanger.ac.uk/pub2/wormbase/releases/WS230/
species/h_contortus/h_contortus.WS230.protein.fa.gz.
Homo sapiens: ftp://ftp.ensembl.org/pub/release-70/fasta/homo_sapiens/pep/Homo_sapiens.
GRCh37.70.pep.all.fa.gz, dated 12/19/12.
Meloidogyne hapla (parasitic nematode of plants): ftp://ftp.sanger.ac.uk/pub2/wormbase/
releases/WS230/species/m_hapla/m_hapla.WS230.protein.fa.gz.
Loa loa (parasitic nematode of humans): loa_loa_v3_3_proteins.fasta, manually downloaded
from http://www.broadinstitute.org/annotation/genome/filarial_worms/MultiDownloads.html.
Meloidogyne incognita (parasitic nematode of plants): http://www.inra.fr/meloidogyne_{—}
incognita/content/download/3010/29690/version/2/file/MincV1A1.fas.
Mus musculus: ftp://ftp.ensembl.org/pub/release-70/fasta/mus_musculus/pep/Mus_musculus.
GRCm38.70.pep.all.fa.gz, dated 12/19/12.
NEMBASE4, translated EST set from diverse nematode species, including the human hookworm
parasite Necator americanus: http://www.nematodes.org/downloads/databases/NEMBASE4/
NEMBASE4_pro.fsa.tgz.
Oesophasostomum dentatum, translated ESTs (parasitic nematode of pigs): http://nematode.net/
Data/transcript_assembly_ftp/O.dentatum_p4ePro.fsa.
Ostertagia ostertagi, translated ESTs (parasitic nematode of cattle): http://nematode.net/Data/
transcript_assembly_ftp/Ostertagia_ostertagi.p4ePro.fsa; and http://nematode.net/Data/
translations_ftp/OS.trans.final.faa.
Parastrongyloides trichosuri, translated ESTs (parasitic nematode of opossums): http://
nematode.net/Data/translations_ftp/PT.trans.final.faa.
Teladorsagia circumcincta, translated ESTs (parasitic nematode of sheep): http://nematode.net/
Data/transcript_assembly_ftp/T.circumcincta_pro.faa.
Toxocara canis, translated ESTs (parasitic nematode): http://nematode.net/Data/translations_ftp/
TX.trans.final.faa.
Trichostrongylus colubriformis, translated ESTs (parasitic nematode): http://nematode.net/Data/
transcript_assembly_ftp/T.colubriformis_p4ePro.fsa.
Pristionchus pacificus (free-living nematode, closely related to both A. ceylanicum and C.
elegans): ftp://ftp.sanger.ac.uk/pub2/wormbase/releases/WS230/species/p_pacificus/p_pacificus.
WS230.protein.fa.gz.
Strongyloides ratti (parasitic nematode of rats): ftp://ftp.sanger.ac.uk/pub2/wormbase/releases/
WS230/species/s_ratti/s_ratti.WS230.protein.fa.gz.
Trichinella spiralis (parasitic nematode of mammals): ftp://ftp.sanger.ac.uk/pub2/wormbase/
releases/WS230/species/t_spiralis/t_spiralis.WS230.protein.fa.gz.
Wuchereria bancrofti (parasitic nematode of humans): wuchereria_bancrofti_1_proteins.fasta,
manually downloaded from http://www.broadinstitute.org/annotation/genome/filarial_worms/
MultiDownloads.html.

To link protein traits (motifs or orthology groups) to biological steps of hookworm infection, the rank-sum statistics for expression levels of genes encoding each trait were calculated. If a set of genes sharing a common protein trait was highly skewed towards genes with upregulation or downregulation between steps of infection, this was detectable by a low rank-sum p-value (≦10⁻⁶) for that set. Genes were ranked by their ratios of expression (later stage/earlier stage), with expression measured in transcripts per million (TPM) by RSEM 1.2.0 (Li and Dewey, 2011); distributions of each protein trait were assessed separately with the Perl module Statistics-Test-WilcoxonRankSum-0.0.7 (from cpan.org).

Among groups of genes significantly upregulated during the first step of infection (from L3i to 24PI), several were already known to be upregulated during parasitic nematode infection (e.g., transthyretin homologs, peptidases, and ASPs). However, a set of 21 A. ceylanicum genes were defined only by an OrthoMCL homology group, which was strongly upregulated during early infection in vivo (from L3i to 24PI, p-value 1.7·10⁻⁶). These proteins encoded none of the motifs from Pfam-A or InterPro associated with ASPs (Allergen V5/Tpx-1-related [IPR001283], Allergen V5/Tpx-1-related, conserved site [IPR018244], CAP domain [IPR014044], or CAP [PF00188.21]), yet they shared at least one block of amino acid similarity. Moreover, they were only weakly upregulated when early infection was simulated by 24 hours of hookworm culture medium (from L3i to 24HCM, p-value 0.03); and thus, they would have gone undetected without in vivo analysis. This gene group defines a new gene family, whose collective upregulation was a previously unknown element of early hookworm infection in vivo.

To better define more A. ceylanicum proteins of this new type, a compilation of nematode protein sequences was searched to convergence with psi-BLAST 2.2.26+, and a query sequence chosen from the 21-gene OrthoMCL group ORTHOMCL896.14spp. The nematode protein compilation included all the proteomes listed above for OrthoMCL analysis, as well as ten other nematode proteomes from WormBase WS230 or other databases (Caenorhabditis angaria, C. brenneri, C. japonica, C. remanei, C. sp. 5, C. sp. 11, Loa loa, Meloidogyne incognita, Strongyloides ratti, and Wuchereria bancrofti; Table 1), and partial peptides from translated ESTs of various nematode species in Nematode.net and NemBase4 (Table 1).

Varying the stringency of the psi-BLAST search resulted in varying numbers of genes. At very high stringency (E≦10⁻¹⁵), psi-BLAST converged on 57 genes from A. ceylanicum, none of which encode ASP-associated protein motifs. At more moderate stringency (E≦10⁻⁹), the search converged on 92 A. ceylanicum genes, one of which also encoded a protein motif associated with ASPs. At this stringency, 20 out of 21 members of ORTHOMCL896.14spp were rediscovered. At still more relaxed stringency (E≦10⁻⁶), psi-BLAST converged on 120 A. ceylanicum genes, of which 117 also encoded ASP-associated protein motifs.

Given these results, all members of ORTHOMCL896.14spp, along with other all genes found through psi-BLAST at 10⁻⁹ that lack known ASP motifs to define an ASP-related family, were categorized as ASPRs. By these criteria, A. ceylanicum has 92 ASPR genes. By the same criteria, partial sequences of non-Ancylostoma ASPRs were identified in Necator americanus and Oesophagostomum dentatum. Using a profile generated in the E≦10⁻⁹ psi-BLAST search, the NCBI non-redundant protein database (NCBI-nr) was further searched, which elicited one ASPR, “novel secreted protein 16”, secreted by adult parasitic Heligmosomoides polygyrus bakeri nematodes into their mammalian hosts. ASPRs from both A. ceylanicum and other non-Ancylostoma species are listed in Tables 2 and 3. For the set of 91 ASPRs identified through psi-BLAST at 10⁻⁹ (as opposed to the initial set of 21 ASPRs in OrthoMCL), upregulation in early infection (L3i to 24.PI) was even more significant (p=4.6·10⁻⁹), while upregulation during simulated infection in vitro was negligible (p=0.44).

TABLE 2
A. ceylanicum ASPR Genes.

| Gene | OrthoMCL | Aligned | Secreted | Max a.a. | 24.PI/L3i | 24HCM/L3i |
|---|---|---|---|---|---|---|
| Acey_2012.08.05_0002.g551 | | + | + | 138 | 0.8873 | 1.0986 |
| Acey_2012.08.05_0004.g1889 | | + | + | 153 | 10.7576 | 6.3939 |
| Acey_2012.08.05_0005.g2681 | | + | | 179 | 0.7692 | 1.0769 |
| Acey_2012.08.05_0010.g1149 | | + | + | 144 | 0.4080 | 0.5360 |
| Acey_2012.08.05_0012.g1803 | | | + | 136 | 6.5657 | 1.3283 |
| Acey_2012.08.05_0013.g2037 | | | + | 130 | 0.6119 | 1.5672 |
| Acey_2012.08.05_0013.g2039 | | + | + | 144 | 0.0440 | 0.2622 |
| Acey_2012.08.05_0015.g2843 | | | | 205 | 0.7273 | 2.1818 |
| Acey_2012.08.05_0015.g2844 | | | + | 149 | 4023.7647 | 7.2353 |
| Acey_2012.08.05_0015.g2860 | | + | + | 151 | 0.8125 | 1.0625 |
| Acey_2012.08.05_0015.g2865 | + | | + | 198 | 8.8333 | 13.3333 |
| Acey_2012.08.05_0015.g2877 | + | + | + | 144 | 10.4412 | 6.4706 |
| Acey_2012.08.05_0015.g2878 | + | | | 223 | 0.8333 | 1.0833 |
| Acey_2012.08.05_0015.g2879 | + | | + | 155 | 6167.8421 | 118.1579 |
| Acey_2012.08.05_0015.g2880 | + | | + | 126 | 5114.0000 | 1270.1719 |
| Acey_2012.08.05_0015.g2881 | + | + | + | 147 | 0.2398 | 0.3216 |
| Acey_2012.08.05_0018.g3499 | + | | | 177 | 1.5385 | 1.0769 |
| Acey_2012.08.05_0018.g3621 | | | | 229 | 1.2955 | 1.0455 |
| Acey_2012.08.05_0020.g126 | | | + | 247 | 12.9615 | 1.0769 |
| Acey_2012.08.05_0020.g128 | | | + | 170 | 5.6129 | 2.1290 |
| Acey_2012.08.05_0020.g130 | | | + | 199 | 14.1221 | 0.6947 |
| Acey_2012.08.05_0020.g40 | | | | 186 | 0.0239 | 0.2840 |
| Acey_2012.08.05_0020.g41 | | | | 104 | 0.1474 | 0.1859 |
| Acey_2012.08.05_0020.g73 | | | | 102 | 1.0360 | 1.1441 |
| Acey_2012.08.05_0020.g74 | | | | 193 | 23.9796 | 1.3061 |
| Acey_2012.08.05_0020.g78 | | | + | 125 | 0.8333 | 2.7143 |
| Acey_2012.08.05_0022.g496 | | | + | 193 | 1.0455 | 1.0682 |
| Acey_2012.08.05_0023.g742 | + | | + | 154 | 17.8125 | 8.3750 |
| Acey_2012.08.05_0025.g1272 | | + | + | 138 | 0.8333 | 1.0556 |
| Acey_2012.08.05_0031.g2245 | | + | + | 147 | 0.8000 | 1.0667 |
| Acey_2012.08.05_0031.g2246 | | | | 101 | 0.0939 | 0.1155 |
| Acey_2012.08.05_0031.g2249 | | | | 80 | 1.0263 | 1.1316 |
| Acey_2012.08.05_0034.g2844 | | + | + | 155 | 218.1250 | 1.5625 |
| Acey_2012.08.05_0039.g111 | | + | + | 158 | 3.1000 | 1.0000 |
| Acey_2012.08.05_0042.g572 | | | + | 187 | 0.7692 | 33.4615 |
| Acey_2012.08.05_0042.g574 | | + | + | 157 | 0.0774 | 135.6258 |
| Acey_2012.08.05_0042.g709 | | | + | 179 | 0.7692 | 1.0769 |
| Acey_2012.08.05_0042.g717 | | | + | 160 | 0.8000 | 8.5333 |
| Acey_2012.08.05_0043.g842 | | + | + | 149 | 0.2419 | 0.8977 |
| Acey_2012.08.05_0045.g1247 | | + | + | 137 | 17.9444 | 1.1111 |
| Acey_2012.08.05_0046.g1415 | | | | 102 | 1.0000 | 1.1343 |
| Acey_2012.08.05_0046.g1417 | | + | + | 130 | 24.6500 | 8.4000 |
| Acey_2012.08.05_0046.g1420 | | | + | 158 | 1.1875 | 1.0625 |
| Acey_2012.08.05_0064.g3511 | | | + | 225 | 1.7692 | 1.0513 |
| Acey_2012.08.05_0067.g109 | | | | 234 | 1.7158 | 1.4806 |
| Acey_2012.08.05_0067.g115 | | | | 174 | 0.1503 | 1.2197 |
| Acey_2012.08.05_0081.g1423 | | | + | 107 | 1.2833 | 1.1000 |
| Acey_2012.08.05_0081.g1426 | | + | + | 143 | 12.1765 | 1.0588 |
| Acey_2012.08.05_0081.g1427 | | + | + | 156 | 0.7500 | 1.0625 |
| Acey_2012.08.05_0081.g1428 | | | | 242 | 0.7692 | 1.0385 |
| Acey_2012.08.05_0081.g1431 | | + | + | 148 | 0.7647 | 1.0588 |
| Acey_2012.08.05_0081.g1435 | | + | + | 147 | 0.8205 | 2.0000 |
| Acey_2012.08.05_0097.g2983 | | | | 162 | 1.5667 | 1.0667 |
| Acey_2012.08.05_0106.g3732 | | | + | 170 | 1.2192 | 0.3733 |
| Acey_2012.08.05_0106.g3734 | | | + | 172 | 44.1290 | 1.8871 |
| Acey_2012.08.05_0120.g903 | | | | 194 | 555.7381 | 188.7143 |
| Acey_2012.08.05_0123.g1117 | | + | + | 135 | 0.8108 | 2.7027 |
| Acey_2012.08.05_0145.g2468 | | | + | 188 | 3771.5273 | 1100.5182 |
| Acey_2012.08.05_0148.g2676 | | | | 103 | 0.8846 | 1.1154 |
| Acey_2012.08.05_0174.g441 | + | | | 82 | 2.0278 | 1.1389 |
| Acey_2012.08.05_0188.g1150 | | | + | 182 | 0.8475 | 0.5847 |
| Acey_2012.08.05_0201.g1711 | + | + | + | 145 | 10.1765 | 2.5647 |
| Acey_2012.08.05_0201.g1712 | + | | | 138 | 74.7742 | 1.0968 |
| Acey_2012.08.05_0210.g2121 | | | | 67 | 1.2041 | 1.2041 |
| Acey_2012.08.05_0233.g3068 | | + | + | 151 | 0.4819 | 0.4217 |
| Acey_2012.08.05_0233.g3072 | | + | + | 147 | 0.8696 | 1.0870 |
| Acey_2012.08.05_0233.g3073 | | | + | 152 | 2.4527 | 9.5878 |
| Acey_2012.08.05_0234.g3130 | | + | + | 159 | 0.7941 | 1.0588 |
| Acey_2012.08.05_0256.g373 | | | | 151 | 0.8000 | 1.0857 |
| Acey_2012.08.05_0258.g456 | | | | 162 | 1.5625 | 1.0312 |
| Acey_2012.08.05_0283.g1303 | | | | 356 | 0.1079 | 0.1715 |
| Acey_2012.08.05_0283.g1311 | | | | 193 | 6.1636 | 4.8727 |
| Acey_2012.08.05_0287.g1455 | | + | + | 144 | 4.4808 | 0.7115 |
| Acey_2012.08.05_0352.g3266 | | | + | 117 | 23.0000 | 6.5909 |
| Acey_2012.08.05_0357.g3396 | | | + | 223 | 6.3243 | 1.0541 |
| Acey_2012.08.05_0457.g1799 | + | + | | 156 | 21.7500 | 2.0625 |
| Acey_2012.08.05_0457.g1800 | + | + | + | 144 | 0.0613 | 0.5339 |
| Acey_2012.08.05_0457.g1803 | + | | | 126 | 5.3922 | 1.2843 |
| Acey_2012.08.05_0457.g1806 | + | + | + | 145 | 8.3529 | 0.5294 |
| Acey_2012.08.05_0457.g1807 | + | + | + | 144 | 21.7059 | 1.0588 |
| Acey_2012.08.05_0457.g1808 | + | + | + | 172 | 16559.0000 | 6.2759 |
| Acey_2012.08.05_0457.g1809 | + | + | + | 157 | 6.9779 | 2.4044 |
| Acey_2012.08.05_0457.g1810 | + | | | 107 | 0.2200 | 0.2700 |
| Acey_2012.08.05_0457.g1812 | + | | | 75 | 26.2439 | 1.1707 |
| Acey_2012.08.05_0457.g1813 | + | | + | 171 | 1.7063 | 0.5238 |
| Acey_2012.08.05_0473.g2085 | | | | 91 | 182.7419 | 1.1290 |
| Acey_2012.08.05_0473.g2087 | | + | + | 141 | 230.6667 | 15.3404 |
| Acey_2012.08.05_0599.g480 | | + | | 172 | 0.0792 | 0.2755 |
| Acey_2012.08.05_0599.g483 | | + | | 133 | 1.0917 | 2.0734 |
| Acey_2012.08.05_0623.g780 | | + | + | 161 | 0.7812 | 1.0312 |
| Acey_2012.08.05_0659.g1258 | | | | 148 | 0.9571 | 0.5322 |
| Acey_2012.08.05_0659.g1260 | | | + | 165 | 0.0839 | 2.6642 |

For the 92 ASPR genes in A. ceylanicum, the following are noted: which 21 genes were originally found by OrthoMCL; which 36 could be fully aligned with MUSCLE; which 59 genes were predicted to encode secreted proteins by Phobius; the size of their largest product in amino acids; and their ratios of 24PI/L3i and 24HCM/L3i expression. Most ASPR genes are predicted to encode secreted proteins, and the general trend is for much stronger upregulation during in vivo infection than during in vitro simulated infection.

TABLE 3
Identities of non-A. ceylanicum ASPRs.

| Name | Species | Database | Annotation |
|---|---|---|---|
| Hpol-ASPR-nsp-16 | Heligmosomoides | NCBI-nr | gi\|345499006\|emb\|CCC54335.1\| |
| | polygyrus bakeri | | novel secreted protein 16 |
| Nam-ASPR-065676 | Necator americanus | NEMBASE4 | NAP00098_1 nuclear plus strand |
| | | | Method: p4e−>ESTScan |
| Nam-ASPR-102019 | Necator americanus | NEMBASE4 | NAP01298_1 nuclear plus strand |
| | | | Method: p4e−>ESTScan |
| Oden-ASPR-1074 | Oesophagostomum | Nematode.net | Oden_isotig10740 nuclear minus |
| | dentatum | | strand Method: p4e−>ESTScan |
| Oden-ASPR-10741 | Oesophagostomum | Nematode.net | Oden_isotig10741 nuclear minus |
| | dentatum | | strand Method: p4e−>ESTScan |
| Oden-ASPR-10742 | Oesophagostomum | Nematode.net | Oden_isotig10742 nuclear minus |
| | dentatum | | strand Method: p4e−>ESTScan |
| Oden-ASPR-12576 | Oesophagostomum | Nematode.net | Oden_isotig12576 nuclear plus |
| | dentatum | | strand Method: p4e−>ESTScan |
| Oden-ASPR-12577 | Oesophagostomum | Nematode.net | Oden_isotig12577 nuclear plus |
| | dentatum | | strand Method: p4e−>ESTScan |
| Oden-ASPR-12578 | Oesophagostomum | Nematode.net | Oden_isotig12578 nuclear plus |
| | dentatum | | strand Method: p4e−>ESTScan |
| Oden-ASPR-22809 | Oesophagostomum | Nematode.net | Oden_isotig22809 nuclear minus |
| | dentatum | | strand Method: p4e−>ESTScan |
| Oden-ASPR-23562 | Oesophagostomum | Nematode.net | Oden_isotig23562 nuclear plus |
| | dentatum | | strand Method: p4e−>ESTScan |
| Oden-ASPR-24342 | Oesophagostomum | Nematode.net | Oden_isotig24342 nuclear plus |
| | dentatum | | strand Method: p4e−>ESTScan |
| Oden-ASPR-24659 | Oesophagostomum | Nematode.net | Oden_isotig24659 nuclear plus |
| | dentatum | | strand Method: p4e−>ESTScan |
| Oden-ASPR-25419 | Oesophagostomum | Nematode.net | Oden_isotig25419 nuclear plus |
| | dentatum | | strand Method: p4e−>ESTScan |

To better define the relationship between ASPRs and ASPs, 91 A. ceylanicum ASPRs were aligned with MUSCLE 3.8.31, and JalView 2.8 was used to select a subset with full-length alignments. In parallel, 499 ASP genes in A. ceylanicum were found to encode one or more of the ASP-associated motifs from Pfam-A or InterPro. As with ASPRs, these 499 ASP genes were aligned, and a subset was selected with full-length alignments. This yielded a group of 36 ASPRs (Table 2) and 235 ASPs from A. ceylanicum that formed full-length alignments. These fully-alignable subsets of ASPRs and ASPs were aligned together, which in turn allows the construction of an evolutionary tree relating these two gene families.

### Example 2

### Identification of Ancylostoma ceylanicum Genes Related to Mammalian Genes

Example 2 describes a set of genes in Ancylostoma ceylanicum with the following traits, which indicate that their products either might be useful vaccines: their gene products resemble mammalian proteins with immunological functions; they have likely been retained because they confer advantages during parasitism; and several of the genes are strongly upregulated during the establishment of mature infection, between 5 and 12 days after A. ceylanicum infects its host. In one case, an analogous gene is present in the genome of the roundworm Ascaris suum, a close relative of the human parasite A. lumbricoides, and thus is a particularly strong vaccine candidate for both A. ceylanicum and A. lumbricoides. The predicted coding DNA sequences for these A. ceylanicum genes are disclosed in SEQ ID NO:188 to SEQ ID NO:203, which encode amino acid sequences that may serve as useful antigens for preventing or treating a hookworm infection.

OrthoMCL 1.3 was used to make comparisons of the predicted A. ceylanicum protein-coding genes to those of nine other nematodes (Ascaris suum, Brugia malayi, Bursaphelenchus xylophilus, Caenorhabditis briggsae, Caenorhabditis elegans, Dirofilaria immitis, Meloidogyne hapla, Pristionchus pacificus, and Trichinella spiralis) and those of two mammals (Homo sapiens and Mus musculus). Sources of the proteomes that were examined are listed in Table 4.

TABLE 4
Sources of the nematode and mammalian proteomes that were examined

Ancylostoma ceylanicum (zoonotic hookworm parasite): Proprietary; see Sequence Listing
Ascaris suum (roundworm parasite of pigs, closely related to the human roundworm parasite Ascaris
lumbricoides): ftp://ftp.sanger.ac.uk/pub2/wormbase/releases/WS230/species/a_suum/a_suum.WS230.
protein.fa.gz.
Brugia malayi (parasitic nematode of humans): ftp://ftp.sanger.ac.uk/pub2/wormbase/releases/WS230/
species/b_malayi/b_malayi.WS230.protein.fa.gz.
Bursaphelenchus xylophilus (parasitic nematode of trees): ftp://ftp.sanger.ac.uk/pub2/wormbase/releases/
WS230/species/b_xylophilus/b_xylophilus.WS230.protein.fa.gz.
Caenorhabditis briggsae (non-parasitic nematode, closely related to C. elegans): ftp://ftp.sanger.ac.uk/
pub2/wormbase/releases/WS230/species/c_briggsae/c_briggsae.WS230.protein.fa.gz.
Caenorhabditis elegans (experimentally well-characterized non-parasitic nematode): ftp://ftp.sanger.ac.
uk/pub2/wormbase/releases/WS230/species/c_elegans/c_elegans.WS230.protein.fa.gz.
Dirofilaria immitis (parasitic nematode of dogs): http://nematodes.org/downloads/959nematodegenomes/
blast/db2/nDi.2.2.2.aug.proteins.fasta.gz, dated 07-Aug-2012 12:26.
Homo sapiens: ftp://ftp.ensembl.org/pub/release-70/fasta/homo_sapiens/pep/Homo_sapiens.GRCh37.70.
pep.all.fa.gz, dated 12/19/12.
Meloidogyne hapla (parasitic nematode of plants): ftp://ftp.sanger.ac.uk/pub2/wormbase/releases/WS230/
species/m_hapla/m_hapla.WS230.protein.fa.gz.
Mus musculus: ftp://ftp.ensembl.org/pub/release-70/fasta/mus_musculus/pep/Mus_musculus.GRCm38.
70.pep.all.fa.gz, dated 12/19/12.
Pristionchus pacificus (free-living nematode, closely related to both A. ceylanicum and C. elegans): ftp://
ftp.sanger.ac.uk/pub2/wormbase/releases/WS230/species/p_pacificus/p_pacificus.WS230.protein.fa.gz.
Trichinella spiralis (parasitic nematode of mammals): ftp://ftp.sanger.ac.uk/pub2/wormbase/releases/
WS230/species/t_spiralis/t_spiralis.WS230.protein.fa.gz.

The A. ceylanicum genes were assessed to determine if any had relatedness with both humans and mice, or with the animal parasites A. suum, B. malayi, D. immitis, or T. spiralis, but not with the free-living nematodes C. elegans, C. briggsae, or P. pacificus (all of which are much more closely related to A. ceylanicum than A. suum), nor with the plant-parasitic nematodes B. xylophilus or M. hapla. Out of 33,243 groups, 52 were identified as similar to mammalian genes. The A. ceylanicum proteins were further examined by BlastP searches of the NCBI non-redundant (NCBI-nr) protein database. In most cases, BlastP showed similarities to C. elegans and other nematodes, and these genes were not considered further. However, eight A. ceylanicum genes were identified as related to vertebrate genes while having no non-parasitic nematode orthologs (Table 5). They fall into three classes based on their most obvious similarities to mammalian proteins: mannose receptors; asialoglycoprotein receptors; and a variety of lectins. Strikingly, all three classes of similarities are to mammalian proteins with C-lectin domains, which are generally involved in binding glycoproteins, endocytosis, and immunological responses.

TABLE 5
Genes in A. ceylanicum and their similarities to mammalian or deuterostome genes.

| | | Size of largest | Up regulation | |
|---|---|---|---|---|
| | Mammalian | isoform | from day | SEQ ID |
| A. ceylanicum gene | similarity | (residues) | 5 to 12 | NO(s) |
| Acey_2012.08.05_0010.g910 | Mannose receptor | 895 | 323-fold | 190; 191; |
| | | | | 192 |
| Acey_2012.08.05_0230.g2988 | Mannose receptor | 869 | 46-fold | 200; 201 |
| Acey_2012.08.05_0004.g2039 | Asialoglycoprotein | 126 | 4-fold | 189 |
| | receptor | | | |
| Acey_2012.08.05_0004.g1962 | Asialoglycoprotein | 166 | 91-fold | 188 |
| | receptor | | | |
| Acey_2012.08.05_0065.g3635 | [Asialoglycoprotein | 291 | 8-fold | 194; 195 |
| | receptor, but weak | | | |
| | match] | | | |
| Acey_2012.08.05_0010.g996 | Neurocan and other | 157 | 154-fold | 193 |
| | chondroitin sulfate | | | |
| | proteoglycans | | | |
| Acey_2012.08.05_0212.g2239 | Macrophage | 224 | 45-fold | 196; 197; |
| | asialoglycoprotein- | | | 198; 199 |
| | binding protein 1- | | | |
| | like, CD209 antigen- | | | |
| | like protein 2-like, | | | |
| | neurocan, etc. | | | |
| Acey_2012.08.05_0517.g2812 | Vertebrate lectin | 165 | 46-fold | 202; 203 |
| | proteins | | | |

To further determine their possible relevance to infection, the expression profile for all of these A. ceylanicum genes was assessed by RNA-seq analysis at the following infection stages (with A. ceylanicum in golden hamsters): infectious third-stage larvae, before infection (L3i); 24 hours after infection in vivo (in the stomach lining of the hamster); 24 hours after incubation in hookworm culture medium, a commonly used synthetic model of infection; 5 days after infection (5.D); 12 days after infection (12.D); 17 and 19 days after infection (17.D and 19.D). The expression of six of these genes was strongly upregulated from 5.D to 12.D, and remained high thereafter (Table 5). This pattern was found for both mannose receptor-like genes, one asialoglycoprotein receptor-like gene, and three lectin-like genes.

The mannose receptor-like genes Acey_{—}2012.08.05_{—}0010.g910 and Acey_{—}2012.08.05_{—0230.}g2988 are similar to mammalian mannose receptors (as indicated by BlastP searches and their general organization, with N-terminal signal sequences for secretion followed by five C-lectin domains). In mammals, mannose receptors are expressed in macrophages, are required for normal clearance of glycoproteins, and they are thought to modulate immune responses to fungi and helminths. These receptors belong to a larger superfamily of receptor proteins with four well-known families (mannose receptors MRC1 and MRC2; lymphocyte antigen LY75; and secretory phospholipase A2 receptor PLA2R), along with a fifth family of non-vertebrate deuterostome MRC-like proteins (from acorn worms, lancelets, sea urchins, and sea squirts), termed “MRCL” herein.

### Example 3

### Identification of Ancylostoma ceylanicum Genes that are likely necessary to sustain a Ancylostoma ceylanicum Infection

Example 3 describes a strategy for identifying those proteins in a parasite genome which are most likely to be generally efficacious, parasite-specific drug targets, and thus, their amino acid sequences may comprise suitable antigens for use in a vaccine. Specifically, those proteins encoded by the Ancylostoma ceylanicum genome are identified that have the following traits: a reasonable likelihood of being inhibited by drugs (“druggable”) and an associated three-dimensional protein structure (enabling rational drug design); required for normal biological function in the experimental nematode Caenorhabditis elegans (with mutant phenotypes, indicating both that the proteins are likely to be required for survival of A. ceylanicum and that assaying the drugs in C. elegans will be straightforward); present in the genome of Ascaris suum, a close relative of the human parasite A. lumbricoides (so that a drug effective against an A. ceylanicum target might also be effective against an A. lumbricoides target, in a human infected with both hookworms and roundworms); absent from the genomes of Homo sapiens and Mus musculus (so that drugs against these proteins are less likely to harm humans or other mammals being treated by the drugs); and, optionally, present in other parasites (so that drugs may have very broad applicability). The identities of the predicted target motifs are disclosed, with predicted coding DNA sequences (SEQ ID NO:204 to SEQ ID NO:404) of the resulting A. ceylanicum target gene products, which encode amino acid sequences that may serve as useful antigens for preventing or treating a hookworm infection.

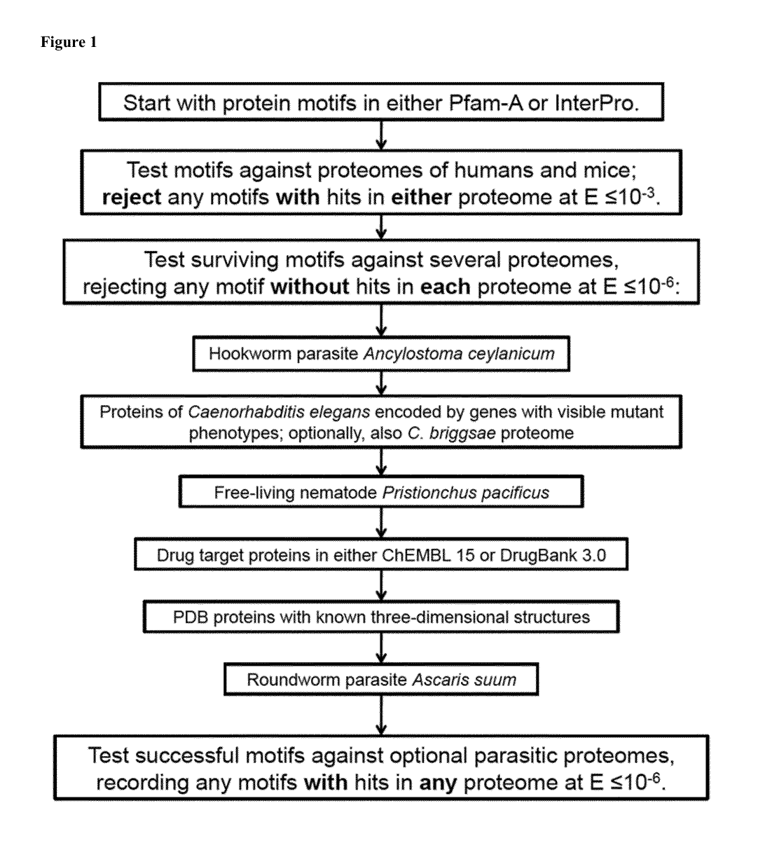

The proteome of Ancylostoma ceylanicum was scanned along with a number of other proteomes for instances of protein motifs using two search programs and motif databases: the HMMER 3.0 program with the Pfam-A 26.0 motif database; and the InterProScan 4.8 program with its associated motif database (which includes several public databases) (see FIG. 1).

First, banned proteomes were searched for instances of motifs, counting any motif to exist in that proteome if it occurred with an E-value of ≦10⁻³. The banned proteomes were the ENSEMBL sequences from release 70 for human beings (Homo sapiens) and mice (Mus musculus). Sources for these and other proteomes are listed in Table 6. Any motif from either Pfam-A or InterPro was disqualified if it had a hit in either banned proteome. Since H. sapiens and M. musculus were the first two mammalian genomes to be sequenced, their gene predictions are of exceptionally high quality; any gene conserved in mammals generally is thus likely to be effectively annotated in either humans or mice, and to be detected in the screen.

TABLE 6
Sources of protein sequences and, where noted, their documentation

Banned proteomes:
Homo sapiens: ftp://ftp.ensembl.org/pub/release-70/fasta/homo_sapiens/pep/Homo_sapiens.GRCh37.70.
pep.all.fa.gz, dated 12/19/12.
Mus musculus: ftp://ftp.ensembl.org/pub/release-70/fasta/mus_musculus/pep/Mus_musculus.GRCm38.
70.pep.all.fa.gz, dated 12/19/12.
Required proteomes:
Ancylostoma ceylanicum (zoonotic hookworm parasite): Proprietary; see Sequence Listing
Ascaris suum (roundworm parasite of pigs, closely related to the human roundworm parasite Ascaris
lumbricoides): ftp://ftp.sanger.ac.uk/pub2/wormbase/releases/WS230/species/a_suum/a_suum.WS230.
protein.fa.gz.
Caenorhabditis briggsae (required only for HMMER 3/Pfam-A searches; non-parasitic nematode, closely
related to C. elegans): ftp://ftp.sanger.ac.uk/pub2/wormbase/releases/WS230/species/c_briggsae/
c_briggsae.WS230.protein.fa.gz.
Caenorhabditis elegans (experimentally well-characterized non-parasitic nematode): ftp://ftp.sanger.ac.
uk/pub2/wormbase/releases/WS230/species/c_elegans/c_elegans.WS230.protein.fa.gz.
The subset of the C. elegans proteins associated with genes having mutant phenotypes in WormBase
WS220 was selected for motif scanning.
ChEMBL 15 (known drug targets): ftp://ftp.ebi.ac.uk/pub/databases/chembl/ChEMBLdb/releases/
chembl_15/chembl_15.fa.gz; dated 1/30/13, 2:14:00 PM.
Documentation: ftp://ftp.ebi.ac.uk/pub/databases/chembl/ChEMBLdb/releases/chembl_15/README,
dated 2/12/13, 9:16:00 AM; ftp://ftp.ebi.ac.uk/pub/databases/chembl/ChEMBLdb/releases/chembl_15/
chembl_15_release_notes.txt, dated 1/30/13 2:36:00 PM; and ftp://ftp.ebi.ac.uk/pub/databases/chembl/
ChEMBLdb/releases/chembl_15/chembl_15_mysql.tar.gz, dated 1/30/13, 2:14:00 PM.
DrugBank 3.0 (known drug targets): All protein sequences: http://www.drugbank.ca/system/downloads/
current/sequences/protein/all_target.fasta.zip; dated 2012-10-21 08:00.
Withdrawn drug targets: http://www.drugbank.ca/system/downloads/current/sequences/protein/
withdrawn_target.fasta.zip; dated 2012-10-21 08:00.
The subset of non-withdrawn drug targets (found in all_target.fasta but not in withdrawn_target.fasta) was
selected from all_target.fasta, and then scanned with motifs.
Documentation: http://www.drugbank.ca/system/downloads/current/drugbank.txt.zip; dated 2012-10-21
01:09.
PDB (proteins with known three-dimensional structures): ftp://ftp.ncbi.nlm.nih.gov/blast/db/FASTA/
pdbaa.gz, dated 2/19/13, 8:22:00 AM.
Pristionchus pacificus (free-living nematode, closely related to both A. ceylanicum and C. elegans): ftp://
ftp.sanger.ac.uk/pub2/wormbase/releases/WS230/species/p_pacificus/p_pacificus.WS230.protein.fa.gz.
Optional proteomes:
Brugia malayi (parasitic nematode of humans): ftp://ftp.sanger.ac.uk/pub2/wormbase/releases/WS230/
species/b_malayi/b_malayi.WS230.protein.fa.gz.
Cryptosporidium parvum (protozoan parasite, causes cryptosporidiosis): http://cryptodb.org/common/
downloads/release-5.0/CparvumIowaII/fasta/data/CryptoDB-5.0_CparvumIowaII_AnnotatedProteins.
fasta, dated 30-Jun-2012 08:12.
Dirofilaria immitis (parasitic nematode of dogs): http://nematodes.org/downloads/959nematodegenomes/
blast/db2/nDi.2.2.2.aug.proteins.fasta.gz, dated 07-Aug-2012 12:26.
Encephalitozoon cuniculi (fungus, intracellular parasite, harmful to immunocompromised humans): http://
microsporidiadb.org/common/downloads/release-3.0/EcuniculiEC1/fasta/data/MicrosporidiaDB-3.
0_EcuniculiEC1_AnnotatedProteins.fasta, dated 30-Jun-2012 08:16.
Entamoeba histolytica (protozoan parasite, causes amoebiasis): http://amoebadb.org/common/downloads/
release-1.7/EhistolyticaHM-1:IMSS/fasta/EhistolyticaHM-1:IMSSAnnotatedProteins_AmoebaDB-1.7.
fasta, dated 30-Jun-2012 08:08.
Giardia lamblia (protozoan parasite, causes giardiasis): http://giardiadb.org/common/downloads/release-
2.5/GintestinalisAssemblageA/fasta/GintestinalisAssemblageAAnnotatedProteins_GiardiaDB-2.5.fasta,
dated 30-Jun-2012 08:13.
Leishmania major (protozoan parasite, causes leishmaniasis): http://tritrypdb.org/common/downloads/
release-4.2/Lmajor/fasta/LmajorFriedlinAnnotatedProteins_TriTrypDB-4.2.fasta, dated 15-Aug-2012
12:54.
Meloidogyne hapla (parasitic nematode of plants): ftp://ftp.sanger.ac.uk/pub2/wormbase/releases/WS230/
species/m_hapla/m_hapla.WS230.protein.fa.gz.
Neospora caninum (protozoan parasite, causes spontaneous abortion in livestock): http://toxodb.org/
common/downloads/release-8.0/NcaninumLIV/fasta/data/ToxoDB-8.
0_NcaninumLIV_AnnotatedProteins.fasta, dated 07-Sep-2012 13:51.
Plasmodium falciparum (protozoan parasite, causes malaria): http://plasmodb.org/common/downloads/
release-9.2/Pfalciparum3D7/fasta/data/PlasmoDB-9.2_Pfalciparum3D7_AnnotatedProteins.fasta, dated
23-Oct-2012 15:18.
Plasmodium vivax (protozoan parasite, causes malaria): http://plasmodb.org/common/downloads/release-
9.2/PvivaxSaI1/fasta/data/PlasmoDB-9.2_PvivaxSaI1_AnnotatedProteins.fasta, dated 15-Oct-2012 15:16.
Schistosoma japonicum (trematode parasite, causes schistosomiasis): http://www.chgc.sh.cn/japonicum/
resource/GeneDB_Sjaponicum.v4.zip, dated 04-Jun-2009 14:54.
Schistosoma mansoni (trematode parasite, causes schistosomiasis): ftp://ftp.sanger.ac.uk/pub/pathogens/
Schistosoma/mansoni/genome/gene_predictions/GeneDB_Smansoni_Proteins.v4.0h.gz, dated 8/12/09.
Toxoplasma gondii (intracellular protozoan parasite): http://toxodb.org/common/downloads/release-8.0/
TgondiiME49/fasta/data/ToxoDB-8.0_TgondiiME49_AnnotatedProteins.fasta, dated 07-Sep-2012 13:51.
Theileria annulata (protozoan parasite, causes tropical theileriosis in livestock): ftp://ftp.sanger.ac.uk/
pub/pathogens/T_annulata/TANN.GeneDB.pep, dated 7/15/05.
Trichinella spiralis (parasitic nematode of mammals): ftp://ftp.sanger.ac.uk/pub2/wormbase/releases/
WS230/species/t_spiralis/t_spiralis.WS230.protein.fa.gz.
Trichomonas vaginalis (protozoan parasite, causes trichomoniasis): http://trichdb.org/common/
downloads/release-1.3/Tvaginalis/fasta/TvaginalisAnnotatedProteins_TrichDB-1.3.fasta, dated 30-Jun-2012 08:27
Trypanosoma brucei (protozoan parasite, causes sleeping sickness): http://tritrypdb.org/common/
downloads/release-4.2/Tbrucei/fasta/Tbrucei427AnnotatedProteins_TriTrypDB-4.2.fasta, dated 15-Aug-
2012 12:56.
Trypanosoma cruzi (protozoan parasite, causes Chagas disease): http://tritrypdb.org/common/downloads/
release-4.2/Tcruzi/fasta/TcruziEsmeraldo-LikeAnnotatedProteins_TriTrypDB-4.2.fasta, dated 15-Aug-
2012 12:57.

Required proteomes were searched for instances of motifs, counting any motif to exist in that proteome if it occurred with an E-value of ≦10⁻⁶. Any motif which had not already been detected in a banned proteome with E≦10⁻³ was further considered if and only if this motif was detected in all required proteomes at E≦10⁻⁶. The difference between E values for banned versus required was chosen to ensure that false negatives in the banned proteomes were unlikely.

The required proteomes, for both Pfam-A and InterPro, included the following: A. ceylanicum (nematode, hookworm parasite of humans; sequences taken from the genome analysis); the subset of the C. elegans proteome encoded by genes with mutant phenotypes; Pristionchus pacificus (a free-living experimental nematode like C. elegans, closely related to both A. ceylanicum and C. elegans); ChEMBL 15 or DrugBank 3.0, pooled into a single set of proteins for this analysis (a hit in either contributor to the set was qualifying); the PDB collection of proteins with solved three-dimensional structures; and Ascaris suum (closely related to the human roundworm parasite A. lumbricoides). To accelerate the searches, which for InterProScan could be lengthy, the largest isoform for each gene was generally selected in each proteome. For HMMER 3/Pfam-A searches, Caenorhabditis briggsae (a relative of C. elegans) was also included as a required proteome.

The subset of the C. elegans proteome encoded by genes with mutant phenotypes was chosen to restrict instances of motifs to those proteins in C. elegans, which are demonstrably required for its biological fitness in vivo. Protein sequences were taken from the WS230 release of WormBase. Mutant phenotypes were taken from the WS220 release of WormBase (which was the latest release for which downloadable phenotypes were available at the time of the analysis) and mapped to their WS230 products by WBGene identifiers. Lethal phenotypes were not required because anthelmintic drugs can be effective without having an overtly lethal phenotype. For instance, the widely used ivermectin class of drugs affects glutamate-gated chloride channels and thus produces paralysis rather than immediate death; yet ivermectins are effective against parasitic nematodes, presumably because they cannot survive in their hosts unless their nervous systems are working normally.

Another feature of the motif search step, which biased it towards functionally vital proteins, was that motifs were required to exist in at least four different nematode species. Although the limitation does not guarantee that the presence of such a motif is required generally for nematode survival, it does select against any motif easily dispensable for it.

Third, optional proteomes were searched for instances of motifs which passed both tests above. Optional proteomes were taken from other helminth or protozoan parasites of biomedical significance. These included the following parasitic nematodes: Brugia malayi and Trichinella spiralis. They also included the following trematodes: Schistosoma japonicum and Schistosoma mansoni. Finally, they included the following protozoans: Cryptosporidium parvum; Encephalitozoon cuniculi; Entamoeba histolytica; Giardia lamblia; Leishmania major; Neospora caninum; Plasmodium falciparum; Plasmodium vivax; Toxoplasma gondii; Theileria annulata; Trichomonas vaginalis; Trypanosoma brucei, and Trypanosoma cruzi. For HMMER 3/Pfam-A only, they further included the parasitic nematodes Dirofilaria immitis and Meloidogyne hapla. In all cases, a motif counted as occurring in an optional proteome if it had an E-value of ≦10⁻⁶.

The resulting motif hits for Pfam-A and InterPro are summarized in Tables 7 and 8, and the predicted A. ceylanicum genes encoding the motifs are listed in Tables 9 and 10.

TABLE 7
PFAM domains selected as indicating possible drug targets

| | | Present in | Present in |
|---|---|---|---|
| PFAM | | obligatory | optional |
| accession no. | Motif name | species | species |
| PF00982.16 | Glyco_transf_20 | A. ceylanicum; | N. caninum; |
| | | C. briggsae; | E. cuniculi; |
| | | C. elegans; | D. immitis; |
| | | P. pacificus; | T. annulata; |
| | | A. suum | T. spiralis; |
| | | | T. gondii; |
| | | | B. malayi; |
| | | | C. parvum |
| PF01674.13 | Lipase_2 | A. ceylanicum; | D. immitis; |
| | | C. briggsae; | B. malayi |
| | | C. elegans; | |
| | | P. pacificus; | |
| | | A. suum | |
| PF02615.9 | Ldh_2 | A. ceylanicum; | T. spiralis; |
| | | C. briggsae; | E. histolytica; |
| | | C. elegans; | S. mansoni; |
| | | P. pacificus; | S. japonicum; |
| | | A. suum | B. malayi |
| PF01493.14 | GXGXG | A. ceylanicum; | T. spiralis; |
| | | C. briggsae; | P. falciparum; |
| | | C. elegans; | P. vivax; |
| | | P. pacificus; | D. immitis; |
| | | A. suum | S. mansoni; |
| | | | S. japonicum; |
| | | | B. malayi |
| PF00463.16 | ICL | A. ceylanicum; | N. caninum; |
| | | C. briggsae; | T. gondii |
| | | C. elegans; | |
| | | P. pacificus; | |
| | | A. suum | |
| PF01274.17 | Malate_synthase | A. ceylanicum; | |
| | | C. briggsae; | |
| | | C. elegans; | |
| | | P. pacificus; | |
| | | A. suum | |
| PF04898.9 | Glu_syn_central | A. ceylanicum; | T. spiralis; |
| | | C. briggsae; | P. falciparum; |
| | | C. elegans; | P. vivax; |
| | | P. pacificus; | D. immitis; |
| | | A. suum | S. mansoni; |
| | | | B. malayi |
| PF06415.8 | iPGM_N | A. ceylanicum; | E. cuniculi; |
| | | C. briggsae; | G. intestinalis; |
| | | C. elegans; | E. histolytica; |
| | | P. pacificus; | T. vaginalis; |
| | | A. suum | T. brucei; |
| | | | D. immitis; |
| | | | T. spiralis; |
| | | | L. major |

TABLE 8
InterPro domains selected as indicating possible drug targets

| | | | Present in | Present in |
|---|---|---|---|---|
| Subsidiary | | | obligatory | optional |
| database | Accession no. | Motif name | species | species |
| HMMPanther | PTHR10788 | TREHALOSE-6- | A. ceylanicum; | N. caninum; |
| | | PHOSPHATE SYNTHASE | C. elegans; | E. cuniculi; |
| | | | P. pacificus; | B. malayi; |
| | | | A. suum | T. gondii; |
| | | | | C. parvum; |
| | | | | T. spiralis; |
| | | | | T. annulata |
| HMMPanther | PTHR11603 | FAMILY NOT NAMED | A. ceylanicum; | T. cruzi; |
| | | | C. elegans; | S. mansoni |
| | | | P. pacificus; | |
| | | | A. suum | |
| HMMPanther | PTHR18945:SF26 | GLUTAMATE-GATED | A. ceylanicum; | B. malayi; |
| | | CHLORIDE CHANNEL | C. elegans; | S. mansoni; |
| | | | P. pacificus; | S. japonicum; |
| | | | A. suum | T. spiralis |
| HMMPfam | PF02615 | Ldh_2 | A. ceylanicum; | B. malayi; |
| | | | C. elegans; | S. mansoni; |
| | | | P. pacificus; | S. japonicum; |
| | | | A. suum | E. histolytica; |
| | | | | T. spiralis |
| HMMPanther | PTHR10314:SF8 | CYSTEINE SYNTHASE | A. ceylanicum; | T. cruzi; |
| | | | C. elegans; | B. malayi; |
| | | | P. pacificus; | T. vaginalis; |
| | | | A. suum | E. histolytica; |
| | | | | L. major |
| HMMTigr | TIGR01139 | cysK: cysteine synthase A | A. ceylanicum; | T. cruzi; |
| | | | C. elegans; | E. histolytica; |
| | | | P. pacificus; | L. major |
| | | | A. suum | |
| Gene3D | G3DSA:1.10.1530.10 | no description | A. ceylanicum; | B. malayi; |
| | | | C. elegans; | S. mansoni; |
| | | | P. pacificus; | S. japonicum; |
| | | | A. suum | E. histolytica; |
| | | | | T. spiralis |
| HMMTigr | TIGR01813 | flavo_cyto_c: | A. ceylanicum; | T. cruzi; |
| | | flavocytochrome c | C. elegans; | B. malayi; |
| | | | P. pacificus; | T. spiralis; |
| | | | A. suum | T. brucei; |
| | | | | L. major |
| HMMPanther | PTHR22893 | FAMILY NOT NAMED | A. ceylanicum; | T. cruzi; |
| | | | C. elegans; | T. vaginalis; |
| | | | P. pacificus; | T. brucei; |
| | | | A. suum | L. major; |
| | | | | G. intestinalis |
| HMMPfam | PF01674 | Lipase_2 | A. ceylanicum; | B. malayi |
| | | | C. elegans; | |
| | | | P. pacificus; | |
| | | | A. suum | |
| HMMPanther | PTHR21266:SF19 | OXIDASE/CHLOROPHYLL | A. ceylanicum; | B. malayi |
| | | SYNTHASE | C. elegans; | |
| | | | P. pacificus; | |
| | | | A. suum | |
| HMMPfam | PF00463 | ICL | A. ceylanicum; | |
| | | | C. elegans; | |
| | | | P. pacificus; | |
| | | | A. suum | |
| HMMPfam | PF01645 | Glu_synthase | A. ceylanicum; | B. malayi; |
| | | | C. elegans; | S. mansoni; |
| | | | P. pacificus; | S. japonicum; |
| | | | A. suum | P. falciparum; |
| | | | | P. vivax; |
| | | | | T. spiralis |
| HMMPanther | PTHR11091 | FAMILY NOT NAMED | A. ceylanicum; | B. malayi; |
| | | | C. elegans; | S. mansoni; |
| | | | P. pacificus; | S. japonicum; |
| | | | A. suum | E. histolytica; |
| | | | | T. spiralis |
| HMMPanther | PTHR11632:SF3 | SUCCINATE | A. ceylanicum; | T. cruzi; |
| | | DEHYDROGENASE 2 | C. elegans; | B. malayi; |
| | | FLAVOPROTEIN | P. pacificus; | T. brucei; |
| | | SUBUNIT | A. suum | L. major |
| HMMPfam | PF01274 | Malate_synthase | A. ceylanicum; | |
| | | | C. elegans; | |
| | | | P. pacificus; | |
| | | | A. suum | |
| HMMPfam | PF01493 | GXGXG | A. ceylanicum; | B. malayi; |
| | | | C. elegans; | S. mansoni; |
| | | | P. pacificus; | S. japonicum; |
| | | | A. suum | P. falciparum; |
| | | | | P. vivax; |
| | | | | T. spiralis |
| HMMPanther | PTHR24076:SF72 | SUBFAMILY NOT | A. ceylanicum; | T. cruzi; |
| | | NAMED | C. elegans; | B. malayi; |
| | | | P. pacificus; | S. mansoni; |
| | | | A. suum | S. japonicum; |
| | | | | T. gondii; |
| | | | | T. brucei; |
| | | | | E. cuniculi; |
| | | | | P. falciparum; |
| | | | | T. spiralis; |
| | | | | T. annulata; |
| | | | | L. major |
| HMMPanther | PTHR21266 | FAMILY NOT NAMED | A. ceylanicum; | B. malayi |
| | | | C. elegans; | |
| | | | P. pacificus; | |
| | | | A. suum | |
| HMMPanther | PTHR21631 | FAMILY NOT NAMED | A. ceylanicum; | N. caninum; |
| | | | C. elegans; | T. cruzi; |
| | | | P. pacificus; | T. gondii |
| | | | A. suum | |
| HMMPanther | PTHR11208:SF8 | KH-DOMAIN RNA | A. ceylanicum; | B. malayi; |
| | | BINDING PROTEIN- | C. elegans; | S. mansoni; |
| | | RELATED | P. pacificus; | S. japonicum; |
| | | | A. suum | T. spiralis |
| HMMPanther | PTHR10169:SF19 | DNA TOPOISOMERASE 2 | A. ceylanicum; | N. caninum; |
| | | | C. elegans; | T. cruzi; |
| | | | P. pacificus; | B. malayi; |
| | | | A. suum | T. gondii; |
| | | | | C. parvum; |
| | | | | T. brucei; |
| | | | | G. intestinalis; |
| | | | | E. cuniculi; |
| | | | | E. histolytica; |
| | | | | P. falciparum; |
| | | | | T. spiralis; |
| | | | | T. annulata; |
| | | | | L. major |
| HMMPanther | PTHR11632:SF5 | SUCCINATE | A. ceylanicum; | N. caninum; |
| | | DEHYDROGENASE 2 | C. elegans; | T. cruzi; |
| | | FLAVOPROTEIN | P. pacificus; | B. malayi; |
| | | SUBUNIT | A. suum | S. mansoni; |
| | | | | S. japonicum; |
| | | | | T. gondii; |
| | | | | P. vivax; |
| | | | | T. brucei; |
| | | | | P. falciparum; |
| | | | | T. spiralis; |
| | | | | T. annulata; |
| | | | | L. major |
| HMMPanther | PTHR11732:SF74 | SUBFAMILY NOT | A. ceylanicum; | N. caninum; |
| | | NAMED | C. elegans; | T. cruzi; |
| | | | P. pacificus; | T. gondii; |
| | | | A. suum | T. brucei; |
| | | | | L. major |
| Gene3D | G3DSA:3.30.1370.60 | no description | A. ceylanicum; | B. malayi; |
| | | | C. elegans; | S. mansoni; |
| | | | P. pacificus; | S. japonicum; |
| | | | A. suum | E. histolytica; |
| | | | | T. spiralis |
| HMMPanther | PTHR21110 | FAMILY NOT NAMED | A. ceylanicum; | B. malayi |
| | | | C. elegans; | |
| | | | P. pacificus; | |
| | | | A. suum | |
| HMMPanther | PTHR24096:SF43 | SUBFAMILY NOT | A. ceylanicum; | B. malayi; |
| | | NAMED | C. elegans; | T. spiralis; |
| | | | P. pacificus; | L. major |
| | | | A. suum | |
| HMMPfam | PF13522 | GATase_6 | A. ceylanicum; | N. caninum; |
| | | | C. elegans; | E. cuniculi; |
| | | | P. pacificus; | B. malayi; |
| | | | A. suum | T. gondii; |
| | | | | P. falciparum; |
| | | | | T. brucei; |
| | | | | L. major |
| Gene3D | G3DSA:2.160.20.60 | no description | A. ceylanicum; | B. malayi; |
| | | | C. elegans; | S. mansoni; |
| | | | P. pacificus; | S. japonicum; |
| | | | A. suum | P. falciparum; |
| | | | | P. vivax; |
| | | | | T. spiralis |
| HMMPanther | PTHR11730 | FAMILY NOT NAMED | A. ceylanicum; | T. cruzi; |
| | | | C. elegans; | B. malayi |
| | | | P. pacificus; | |
| | | | A. suum | |
| HMMPfam | PF04898 | Glu_syn_central | A. ceylanicum; | B. malayi; |
| | | | C. elegans; | S. mansoni; |
| | | | P. pacificus; | P. falciparum; |
| | | | A. suum | P. vivax; |
| | | | | T. spiralis |
| HMMPanther | PTHR23408:SF1 | METHYLMALONYL-COA | A. ceylanicum; | T. spiralis; |
| | | MUTASE | C. elegans; | L. major |
| | | | P. pacificus; | |
| | | | A. suum | |
| superfamily | SSF69336 | Alpha subunit of glutamate | A. ceylanicum; | B. malayi; |
| | | synthase, C-terminal domain | C. elegans; | S. mansoni; |
| | | | P. pacificus; | S. japonicum; |
| | | | A. suum | P. falciparum; |
| | | | | P. vivax; |
| | | | | T. spiralis |
| superfamily | SSF89733 | L-sulfolactate | A. ceylanicum; | B. malayi; |
| | | dehydrogenase-like | C. elegans; | S. mansoni; |
| | | | P. pacificus; | S. japonicum; |
| | | | A. suum | E. histolytica; |
| | | | | T. spiralis |
| Gene3D | G3DSA:3.20.20.360 | no description | A. ceylanicum; | |
| | | | C. elegans; | |
| | | | P. pacificus; | |
| | | | A. suum | |
| HMMPanther | PTHR10788:SF6 | TREHALOSE-6- | A. ceylanicum; | N. caninum; |
| | | PHOSPHATE SYNTHASE | C. elegans; | E. cuniculi; |
| | | | P. pacificus; | B. malayi; |
| | | | A. suum | C. parvum; |
| | | | | T. spiralis; |
| | | | | T. annulata |
| superfamily | SSF51645 | Malate synthase G | A. ceylanicum; | |
| | | | C. elegans; | |
| | | | P. pacificus; | |
| | | | A. suum | |
| HMMTigr | TIGR01136 | cysKM: cysteine synthases | A. ceylanicum; | T. cruzi; |
| | | | C. elegans; | T. vaginalis; |
| | | | P. pacificus; | E. histolytica; |
| | | | A. suum | L. major |

TABLE 9
Drug target-associated PFAM domains
and associated A. ceylanicum genes

| Accession no. | Motif name | A. ceylanicum gene |
|---|---|---|
| PF00463.16 | ICL | Acey_2012.08.05_0003.g1172 |
| PF00982.16 | Glyco_transf_20 | Acey_2012.08.05_0245.g3555 |
| PF00982.16 | Glyco_transf_20 | Acey_2012.08.05_1036.g3463 |
| PF01274.17 | Malate_synthase | Acey_2012.08.05_0003.g1172 |
| PF01274.17 | Malate_synthase | Acey_2012.08.05_0003.g1173 |
| PF01493.14 | GXGXG | Acey_2012.08.05_0223.g2677 |
| PF01674.13 | Lipase_2 | Acey_2012.08.05_0009.g764 |
| PF01674.13 | Lipase_2 | Acey_2012.08.05_0049.g1767 |
| PF01674.13 | Lipase_2 | Acey_2012.08.05_0049.g1854 |
| PF01674.13 | Lipase_2 | Acey_2012.08.05_0101.g3398 |
| PF01674.13 | Lipase_2 | Acey_2012.08.05_0179.g703 |
| PF01674.13 | Lipase_2 | Acey_2012.08.05_0674.g1411 |
| PF02615.9 | Ldh_2 | Acey_2012.08.05_0077.g1140 |
| PF02615.9 | Ldh_2 | Acey_2012.08.05_0099.g3157 |
| PF02615.9 | Ldh_2 | Acey_2012.08.05_0343.g3064 |
| PF02615.9 | Ldh_2 | Acey_2012.08.05_0343.g3065 |
| PF04898.9 | Glu_syn_central | Acey_2012.08.05_0223.g2677 |
| PF06415.8 | iPGM_N | Acey_2012.08.05_0104.g3596 |

TABLE 10
Drug target-associated InterPro domains and associated A. ceylanicum genes

| Subsidiary | | | |
|---|---|---|---|
| database | Accession no. | Motif name | A. ceylanicum gene |
| Gene3D | G3DSA:1.10.1530.10 | no description | Acey_2012.08.05_0077.g1140 |
| Gene3D | G3DSA:1.10.1530.10 | no description | Acey_2012.08.05_0343.g3064 |
| Gene3D | G3DSA:1.10.1530.10 | no description | Acey_2012.08.05_0343.g3065 |
| Gene3D | G3DSA:2.160.20.60 | no description | Acey_2012.08.05_0223.g2677 |
| Gene3D | G3DSA:3.20.20.360 | no description | Acey_2012.08.05_0003.g1172 |
| Gene3D | G3DSA:3.20.20.360 | no description | Acey_2012.08.05_0003.g1173 |
| Gene3D | G3DSA:3.30.1370.60 | no description | Acey_2012.08.05_0077.g1140 |
| Gene3D | G3DSA:3.30.1370.60 | no description | Acey_2012.08.05_0099.g3157 |
| Gene3D | G3DSA:3.30.1370.60 | no description | Acey_2012.08.05_0343.g3064 |
| HMMPanther | PTHR10169:SF19 | DNA TOPOISOMERASE 2 | Acey_2012.08.05_0064.g3495 |
| HMMPanther | PTHR10169:SF19 | DNA TOPOISOMERASE 2 | Acey_2012.08.05_0436.g1438 |
| HMMPanther | PTHR10314:SF8 | CYSTEINE SYNTHASE | Acey_2012.08.05_0002.g728 |
| HMMPanther | PTHR10314:SF8 | CYSTEINE SYNTHASE | Acey_2012.08.05_0491.g2411 |
| HMMPanther | PTHR10788:SF6 | TREHALOSE-6- | Acey_2012.08.05_0042.g665 |
| | | PHOSPHATE SYNTHASE | |
| HMMPanther | PTHR10788:SF6 | TREHALOSE-6- | Acey_2012.08.05_0245.g3555 |
| | | PHOSPHATE SYNTHASE | |
| HMMPanther | PTHR10788:SF6 | TREHALOSE-6- | Acey_2012.08.05_1036.g3463 |
| | | PHOSPHATE SYNTHASE | |
| HMMPanther | PTHR10788 | TREHALOSE-6- | Acey_2012.08.05_0015.g2804 |
| | | PHOSPHATE SYNTHASE | |
| HMMPanther | PTHR10788 | TREHALOSE-6- | Acey_2012.08.05_0042.g665 |
| | | PHOSPHATE SYNTHASE | |
| HMMPanther | PTHR10788 | TREHALOSE-6- | Acey_2012.08.05_0042.g671 |
| | | PHOSPHATE SYNTHASE | |
| HMMPanther | PTHR10788 | TREHALOSE-6- | Acey_2012.08.05_0245.g3555 |
| | | PHOSPHATE SYNTHASE | |
| HMMPanther | PTHR10788 | TREHALOSE-6- | Acey_2012.08.05_1036.g3463 |
| | | PHOSPHATE SYNTHASE | |
| HMMPanther | PTHR11091 | FAMILY NOT NAMED | Acey_2012.08.05_0077.g1140 |
| HMMPanther | PTHR11091 | FAMILY NOT NAMED | Acey_2012.08.05_0099.g3157 |
| HMMPanther | PTHR11091 | FAMILY NOT NAMED | Acey_2012.08.05_0343.g3064 |
| HMMPanther | PTHR11091 | FAMILY NOT NAMED | Acey_2012.08.05_0343.g3065 |
| HMMPanther | PTHR11208:SF8 | KH-DOMAIN RNA | Acey_2012.08.05_0027.g1517 |
| | | BINDING PROTEIN- | |
| | | RELATED | |
| HMMPanther | PTHR11208:SF8 | KH-DOMAIN RNA | Acey_2012.08.05_0217.g2408 |
| | | BINDING PROTEIN- | |
| | | RELATED | |
| HMMPanther | PTHR11208:SF8 | KH-DOMAIN RNA | Acey_2012.08.05_0293.g1600 |
| | | BINDING PROTEIN- | |
| | | RELATED | |
| HMMPanther | PTHR11208:SF8 | KH-DOMAIN RNA | Acey_2012.08.05_0347.g3147 |
| | | BINDING PROTEIN- | |
| | | RELATED | |
| HMMPanther | PTHR11208:SF8 | KH-DOMAIN RNA | Acey_2012.08.05_0363.g3530 |
| | | BINDING PROTEIN- | |
| | | RELATED | |
| HMMPanther | PTHR11603 | FAMILY NOT NAMED | Acey_2012.08.05_0031.g2261 |
| HMMPanther | PTHR11603 | FAMILY NOT NAMED | Acey_2012.08.05_0266.g730 |
| HMMPanther | PTHR11603 | FAMILY NOT NAMED | Acey_2012.08.05_0266.g731 |
| HMMPanther | PTHR11632:SF3 | SUCCINATE | Acey_2012.08.05_0011.g1461 |
| | | DEHYDROGENASE 2 | |
| | | FLAVOPROTEIN | |
| | | SUBUNIT | |
| HMMPanther | PTHR11632:SF5 | SUCCINATE | Acey_2012.08.05_0015.g2818 |
| | | DEHYDROGENASE 2 | |
| | | FLAVOPROTEIN | |
| | | SUBUNIT | |
| HMMPanther | PTHR11730 | FAMILY NOT NAMED | Acey_2012.08.05_0004.g1856 |
| HMMPanther | PTHR11730 | FAMILY NOT NAMED | Acey_2012.08.05_0034.g2831 |
| HMMPanther | PTHR11730 | FAMILY NOT NAMED | Acey_2012.08.05_0086.g1948 |
| HMMPanther | PTHR11730 | FAMILY NOT NAMED | Acey_2012.08.05_0151.g2816 |
| HMMPanther | PTHR11730 | FAMILY NOT NAMED | Acey_2012.08.05_0315.g2266 |
| HMMPanther | PTHR11732:SF74 | SUBFAMILY NOT NAMED | Acey_2012.08.05_0059.g2970 |
| HMMPanther | PTHR11732:SF74 | SUBFAMILY NOT NAMED | Acey_2012.08.05_0059.g2971 |
| HMMPanther | PTHR11732:SF74 | SUBFAMILY NOT NAMED | Acey_2012.08.05_0900.g2951 |
| HMMPanther | PTHR18945:SF26 | GLUTAMATE-GATED | Acey_2012.08.05_0036.g3261 |
| | | CHLORIDE CHANNEL | |
| HMMPanther | PTHR18945:SF26 | GLUTAMATE-GATED | Acey_2012.08.05_0036.g3305 |
| | | CHLORIDE CHANNEL | |
| HMMPanther | PTHR18945:SF26 | GLUTAMATE-GATED | Acey_2012.08.05_0080.g1380 |
| | | CHLORIDE CHANNEL | |
| HMMPanther | PTHR18945:SF26 | GLUTAMATE-GATED | Acey_2012.08.05_0096.g2944 |
| | | CHLORIDE CHANNEL | |
| HMMPanther | PTHR18945:SF26 | GLUTAMATE-GATED | Acey_2012.08.05_0247.g67 |
| | | CHLORIDE CHANNEL | |
| HMMPanther | PTHR18945:SF26 | GLUTAMATE-GATED | Acey_2012.08.05_0348.g3183 |
| | | CHLORIDE CHANNEL | |
| HMMPanther | PTHR18945:SF26 | GLUTAMATE-GATED | Acey_2012.08.05_0348.g3184 |
| | | CHLORIDE CHANNEL | |
| HMMPanther | PTHR18945:SF26 | GLUTAMATE-GATED | Acey_2012.08.05_0445.g1589 |
| | | CHLORIDE CHANNEL | |
| HMMPanther | PTHR18945:SF26 | GLUTAMATE-GATED | Acey_2012.08.05_0455.g1763 |
| | | CHLORIDE CHANNEL | |
| HMMPanther | PTHR18945:SF26 | GLUTAMATE-GATED | Acey_2012.08.05_0455.g1764 |
| | | CHLORIDE CHANNEL | |
| HMMPanther | PTHR21110 | FAMILY NOT NAMED | Acey_2012.08.05_0104.g3596 |
| HMMPanther | PTHR21266 | FAMILY NOT NAMED | Acey_2012.08.05_0189.g1175 |
| HMMPanther | PTHR21266:SF19 | OXIDASE/CHLOROPHYLL | Acey_2012.08.05_0189.g1175 |
| | | SYNTHASE | |
| HMMPanther | PTHR21631 | FAMILY NOT NAMED | Acey_2012.08.05_0003.g1172 |
| HMMPanther | PTHR21631 | FAMILY NOT NAMED | Acey_2012.08.05_0003.g1173 |
| HMMPanther | PTHR22893 | FAMILY NOT NAMED | Acey_2012.08.05_0004.g2159 |
| HMMPanther | PTHR22893 | FAMILY NOT NAMED | Acey_2012.08.05_0019.g3945 |
| HMMPanther | PTHR22893 | FAMILY NOT NAMED | Acey_2012.08.05_0035.g3099 |
| HMMPanther | PTHR22893 | FAMILY NOT NAMED | Acey_2012.08.05_0035.g3101 |
| HMMPanther | PTHR22893 | FAMILY NOT NAMED | Acey_2012.08.05_0035.g3102 |
| HMMPanther | PTHR22893 | FAMILY NOT NAMED | Acey_2012.08.05_0035.g3103 |
| HMMPanther | PTHR22893 | FAMILY NOT NAMED | Acey_2012.08.05_0041.g434 |
| HMMPanther | PTHR22893 | FAMILY NOT NAMED | Acey_2012.08.05_0041.g436 |
| HMMPanther | PTHR22893 | FAMILY NOT NAMED | Acey_2012.08.05_0081.g1458 |
| HMMPanther | PTHR22893 | FAMILY NOT NAMED | Acey_2012.08.05_0081.g1460 |
| HMMPanther | PTHR22893 | FAMILY NOT NAMED | Acey_2012.08.05_0351.g3233 |
| HMMPanther | PTHR22893 | FAMILY NOT NAMED | Acey_2012.08.05_0351.g3234 |
| HMMPanther | PTHR22893 | FAMILY NOT NAMED | Acey_2012.08.05_0351.g3237 |
| HMMPanther | PTHR22893 | FAMILY NOT NAMED | Acey_2012.08.05_0370.g104 |
| HMMPanther | PTHR23408:SF1 | METHYLMALONYL-COA | Acey_2012.08.05_0012.g1610 |
| | | MUTASE | |
| HMMPanther | PTHR24076:SF72 | SUBFAMILY NOT NAMED | Acey_2012.08.05_0476.g2142 |
| HMMPanther | PTHR24096:SF43 | SUBFAMILY NOT NAMED | Acey_2012.08.05_0046.g1352 |
| HMMPanther | PTHR24096:SF43 | SUBFAMILY NOT NAMED | Acey_2012.08.05_0062.g3312 |
| HMMPanther | PTHR24096:SF43 | SUBFAMILY NOT NAMED | Acey_2012.08.05_0064.g3544 |
| HMMPanther | PTHR24096:SF43 | SUBFAMILY NOT NAMED | Acey_2012.08.05_0227.g2815 |
| HMMPanther | PTHR24096:SF43 | SUBFAMILY NOT NAMED | Acey_2012.08.05_0227.g2821 |
| HMMPanther | PTHR24096:SF43 | SUBFAMILY NOT NAMED | Acey_2012.08.05_0288.g1479 |
| HMMPanther | PTHR24096:SF43 | SUBFAMILY NOT NAMED | Acey_2012.08.05_0288.g1482 |
| HMMPanther | PTHR24096:SF43 | SUBFAMILY NOT NAMED | Acey_2012.08.05_0288.g1483 |
| HMMPanther | PTHR24096:SF43 | SUBFAMILY NOT NAMED | Acey_2012.08.05_0478.g2202 |
| HMMPanther | PTHR24096:SF43 | SUBFAMILY NOT NAMED | Acey_2012.08.05_0478.g2203 |
| HMMPfam | PF00463 | ICL | Acey_2012.08.05_0003.g1172 |
| HMMPfam | PF01274 | Malate_synthase | Acey_2012.08.05_0003.g1172 |
| HMMPfam | PF01274 | Malate_synthase | Acey_2012.08.05_0003.g1173 |
| HMMPfam | PF01493 | GXGXG | Acey_2012.08.05_0223.g2677 |
| HMMPfam | PF01645 | Glu_synthase | Acey_2012.08.05_0223.g2677 |
| HMMPfam | PF01674 | Lipase_2 | Acey_2012.08.05_0009.g764 |
| HMMPfam | PF01674 | Lipase_2 | Acey_2012.08.05_0049.g1767 |
| HMMPfam | PF01674 | Lipase_2 | Acey_2012.08.05_0049.g1854 |
| HMMPfam | PF01674 | Lipase_2 | Acey_2012.08.05_0101.g3398 |
| HMMPfam | PF01674 | Lipase_2 | Acey_2012.08.05_0179.g703 |
| HMMPfam | PF01674 | Lipase_2 | Acey_2012.08.05_0674.g1411 |
| HMMPfam | PF02615 | Ldh_2 | Acey_2012.08.05_0077.g1140 |
| HMMPfam | PF02615 | Ldh_2 | Acey_2012.08.05_0099.g3157 |
| HMMPfam | PF02615 | Ldh_2 | Acey_2012.08.05_0343.g3064 |
| HMMPfam | PF02615 | Ldh_2 | Acey_2012.08.05_0343.g3065 |
| HMMPfam | PF04898 | Glu_syn_central | Acey_2012.08.05_0223.g2677 |
| HMMPfam | PF13522 | GATase_6 | Acey_2012.08.05_0021.g387 |
| HMMPfam | PF13522 | GATase_6 | Acey_2012.08.05_0024.g889 |
| HMMPfam | PF13522 | GATase_6 | Acey_2012.08.05_0129.g1478 |
| HMMTigr | TIGR01136 | cysKM: cysteine synthases | Acey_2012.08.05_0002.g728 |
| HMMTigr | TIGR01136 | cysKM: cysteine synthases | Acey_2012.08.05_0491.g2411 |
| HMMTigr | TIGR01139 | cysK: cysteine synthase A | Acey_2012.08.05_0002.g728 |
| HMMTigr | TIGR01139 | cysK: cysteine synthase A | Acey_2012.08.05_0491.g2411 |
| HMMTigr | TIGR01813 | flavo_cyto_c: | Acey_2012.08.05_0011.g1461 |
| | | flavocytochrome c | |
| superfamily | SSF51645 | Malate synthase G | Acey_2012.08.05_0003.g1172 |
| superfamily | SSF51645 | Malate synthase G | Acey_2012.08.05_0003.g1173 |
| superfamily | SSF69336 | Alpha subunit of glutamate | Acey_2012.08.05_0223.g2677 |
| | | synthase, C-terminal domain | |
| superfamily | SSF89733 | L-sulfolactate | Acey_2012.08.05_0077.g1140 |
| | | dehydrogenase-like | |
| superfamily | SSF89733 | L-sulfolactate | Acey_2012.08.05_0099.g3157 |
| | | dehydrogenase-like | |
| superfamily | SSF89733 | L-sulfolactate | Acey_2012.08.05_0343.g3064 |
| | | dehydrogenase-like | |
| superfamily | SSF89733 | L-sulfolactate | Acey_2012.08.05_0343.g3065 |
| | | dehydrogenase-like | |

### Example 4

### Identification of Ancylostoma ceylanicum Protease and Protease Inhibitor Genes

Example 4 describes a repertoire of proteases and protease inhibitors in Ancylostoma ceylanicum. They have the following traits, which indicate that their products might be useful for vaccines: they are strongly upregulated at the onset of A. ceylanicum infection in vivo; and they are evolutionarily specific to worms, rather than being strongly related to proteins in mammals. The DNA sequences for these genes are disclosed in SEQ ID NOS:405-540, which encode amino acid sequences that may serve as useful antigens for preventing or treating a hookworm infection. To identify which genes were specifically activated during early infection, A. ceylanicum expression profiles were assessed by RNA-seq analysis with RSEM 1.2.0 of the following infection stages (with A. ceylanicum in golden hamsters): infectious third-stage larvae, before infection (L3i); 24 hours after infection in vivo (in the stomach lining of the hamster; 24PI); 24 hours after incubation in hookworm culture medium, a commonly used synthetic model of infection (24HCM); 5 days after infection (5.D); 12 days after infection (12.D); 17 and 19 days after infection (17.D and 19.D). Expression levels were calculated in transcripts per million (TPM), which allows gene activities to be measured by a fixed standard and compared impartially between differently developmental stages, conditions, or even different organisms. Genes were ranked by their ratios of expression (later stage TPM/earlier stage TPM).

A. ceylanicum genes were classified both by known protein motifs (through HMMER 3.0/Pfam-A 26 and InterProScan 4.8, and by evolutionary relationships to genes in different species (through OrthoMCL 1.3). For OrthoMCL, the predicted A. ceylanicum protein-coding genes were evolutionarily compared to those of nine other nematodes (Ascaris suum, Brugia malayi, Bursaphelenchus xylophilus, Caenorhabditis elegans, C. briggsae, Dirofilaria immitis, Meloidogyne hapla, Pristionchus pacificus, and Trichinella spiralis), and to those of two mammals from Ensembl release 70 (Homo sapiens and Mus musculus). Sources of the proteomes that were examined are listed in Table 11.

TABLE 11
Sources of the nematode and mammalian proteomes that were
compared to define orthology groups in A. ceylanicum.

Ancylostoma ceylanicum (zoonotic hookworm parasite): Proprietary; see Sequence Listing
Ascaris suum (roundworm parasite of pigs, closely related to the human roundworm parasite Ascaris
lumbricoides): ftp://ftp.sanger.ac.uk/pub2/wormbase/releases/WS230/species/a_suum/a_suum.WS230.
protein.fa.gz.
Brugia malayi (parasitic nematode of humans): ftp://ftp.sanger.ac.uk/pub2/wormbase/releases/WS230/
species/b_malayi/b_malayi.WS230.protein.fa.gz.
Bursaphelenchus xylophilus (parasitic nematode of trees): ftp://ftp.sanger.ac.uk/pub2/wormbase/releases/
WS230/species/b_xylophilus/b_xylophilus.WS230.protein.fa.gz.
Caenorhabditis briggsae (non-parasitic nematode, closely related to C. elegans): ftp://ftp.sanger.ac.uk/
pub2/wormbase/releases/WS230/species/c_briggsae/c_briggsae.WS230.protein.fa.gz.
Caenorhabditis elegans (experimentally well-characterized non-parasitic nematode): ftp://ftp.sanger.ac.
uk/pub2/wormbase/releases/WS230/species/c_elegans/c_elegans.WS230.protein.fa.gz.
Dirofilaria immitis (parasitic nematode of dogs): http://nematodes.org/downloads/959nematodegenomes/
blast/db2/nDi.2.2.2.aug.proteins.fasta.gz, dated 07-Aug-2012 12:26.
Homo sapiens: ftp://ftp.ensembl.org/pub/release-70/fasta/homo_sapiens/pep/Homo_sapiens.GRCh37.70.
pep.all.fa.gz, dated 12/19/12.
Meloidogyne hapla (parasitic nematode of plants): ftp://ftp.sanger.ac.uk/pub2/wormbase/releases/WS230/
species/m_hapla/m_hapla.WS230.protein.fa.gz.
Mus musculus: ftp://ftp.ensembl.org/pub/release-70/fasta/mus_musculus/pep/Mus_musculus.GRCm38.
70.pep.all.fa.gz, dated 12/19/12.
Pristionchus pacificus (free-living nematode, closely related to both A. ceylanicum and C. elegans): ftp://
ftp.sanger.ac.uk/pub2/wormbase/releases/WS230/species/p_pacificus/p_pacificus.WS230.protein.fa.gz.
Trichinella spiralis (parasitic nematode of mammals): ftp://ftp.sanger.ac.uk/pub2/wormbase/releases/
WS230/species/t_spiralis/t_spiralis.WS230.protein.fa.gz.

To link the biological functions of genes to steps of hookworm infection, Pfam-A and InterPro motifs were used to assign Gene Ontology (GO) terms to each A. ceylanicum gene with Blast2GO 2.5 (build 23092011). InterProScan and Blast2GO were performed as in Kumar, 2012 (https://githubcom/sujaikumar/assemblage/blob/master/README-annotationmd#how-to-predict-genes-using-a-two-pass-iterative-maker2-workflow); in particular, for Blast2GO, both InterProScan predictions and BlastP results were used against an animal-specific subset of NCBI's nr database.

Having ranked genes by expression ratios and assigned them GO terms, FUNC 0.4.5 was used to compute which GO terms were significantly overrepresented among genes upregulated from L3i to 24PI. Among the overrepresented GO terms, terms for both proteases and protease inhibitors were observed (Table 12).

TABLE 12
A subset of Gene Ontology (GO) terms disproportionately
associated with genes in A. ceylanicum upregulated
in early infection (from L3i to 24PI).

| ID number | Description | q-value |
|---|---|---|
| GO:0004197 | cysteine-type endopeptidase activity | 3.55271e−15 |
| GO:0004867 | serine-type endopeptidase inhibitor activity | 5.82112e−12 |
| GO:0004222 | metalloendopeptidase activity | 1.09247e−08 |
| GO:0004190 | aspartic-type endopeptidase activity | 1.45201e−06 |
| GO:0008236 | serine-type peptidase activity | 2.32024e−05 |
| GO:0004252 | serine-type endopeptidase activity | 0.000295668 |

At the same time, genes that were significantly upregulated from L3i to 24PI were identified with edgeR 3.0.8, using a set of 406 constitutively expressed genes to estimate a biological dispersion of 0.24339 for gene expression between samples. With this dispersion, 1,146 genes were identified as significantly upregulated (with a q-value of 0.001). In contrast, only 108 genes were observed to be significantly upregulated in hookworm culture medium (i.e., from L3i to 24HCM), indicating the greater ability of infection in vivo to elicit gene activity in A. ceylanicum.

A. ceylanicum genes were identified which had all of the following traits: they were annotated for the GO terms; they were significantly upregulated from L3i to 24PI; and they did not belong to an orthology group that included mammalian proteins (from humans or mice). This yielded a group of 48 genes encoding hookworm-specific, infection-induced proteases (Table 13) and 7 genes encoding hookworm-specific, infection-induced protease inhibitors (Table 14).

TABLE 13
A. ceylanicum genes that are significantly upregulated in early infection (from
L3i to 24PI) and encode proteases that lack obvious homology to mammalian proteins.

| Gene | q-value* | Secreted | GO terms |
|---|---|---|---|
| Acey_2012.08.05_0154.g2957 | 2.5561327735976e−13 | + | cysteine-type endopeptidase activity |
| | | | [GO:0004197] |
| Acey_2012.08.05_0195.g1496 | 9.32379466686754e−13 | + | aspartic-type endopeptidase activity |
| | | | [GO:0004190] |
| Acey_2012.08.05_0183.g967 | 1.10100549778176e−12 | + | aspartic-type endopeptidase activity |
| | | | [GO:0004190] |
| Acey_2012.08.05_0154.g2956 | 1.77119938965992e−12 | | cysteine-type endopeptidase activity |
| | | | [GO:0004197] |
| Acey_2012.08.05_0195.g1499 | 4.051972903713e−11 | + | aspartic-type endopeptidase activity |
| | | | [GO:0004190] |
| Acey_2012.08.05_0154.g2968 | 7.16663927409038e−11 | + | cysteine-type endopeptidase activity |
| | | | [GO:0004197] |
| Acey_2012.08.05_0195.g1495 | 3.54332330455291e−09 | | aspartic-type endopeptidase activity |
| | | | [GO:0004190] |
| Acey_2012.08.05_0081.g1454 | 3.70155439027392e−09 | + | serine-type endopeptidase activity |
| | | | [GO:0004252] |
| Acey_2012.08.05_0195.g1502 | 3.51337433723627e−08 | + | aspartic-type endopeptidase activity |
| | | | [GO:0004190] |
| Acey_2012.08.05_0154.g3018 | 5.90600573076656e−08 | + | cysteine-type endopeptidase activity |
| | | | [GO:0004197] |
| Acey_2012.08.05_0195.g1491 | 8.39644714676855e−08 | | aspartic-type endopeptidase activity |
| | | | [GO:0004190] |
| Acey_2012.08.05_0273.g978 | 1.76487169092424e−07 | | metalloendopeptidase activity |
| | | | [GO:0004222] |
| Acey_2012.08.05_0154.g3016 | 7.51995037789084e−07 | + | cysteine-type endopeptidase activity |
| | | | [GO:0004197] |
| Acey_2012.08.05_0035.g3090 | 1.19541843568679e−06 | | serine-type peptidase activity |
| | | | [GO:0008236] |
| Acey_2012.08.05_0273.g992 | 1.476389860405796−06 | | metalloendopeptidase activity |
| | | | [GO:0004222] |
| Acey_2012.08.05_0619.g730 | 2.76040711398016e−06 | | cysteine-type endopeptidase activity |
| | | | [GO:0004197] |
| Acey_2012.08.05_0154.g2959 | 3.39600112623981e−06 | | cysteine-type endopeptidase activity |
| | | | [GO:0004197] |
| Acey_2012.08.05_0154.g2994 | 5.61369790561758e−06 | | cysteine-type endopeptidase activity |
| | | | [GO:0004197] |
| Acey_2012.08.05_0001.g145 | 1.05261873942301e−05 | + | metalloendopeptidase activity |
| | | | [GO:0004222] |
| Acey_2012.08.05_0258.g432 | 1.33802259147134e−05 | | aspartic-type endopeptidase activity |
| | | | [GO:0004190] |
| Acey_2012.08.05_0007.g3467 | 1.66432960626729e−05 | | cysteine-type endopeptidase activity |
| | | | [GO:0004197] |
| Acey_2012.08.05_0099.g3207 | 2.44344788769675e−05 | + | metalloendopeptidase activity |
| | | | [GO:0004222] |
| Acey_2012.08.05_0004.g1998 | 2.5688646986668e−05 | + | aspartic-type endopeptidase activity |
| | | | [GO:0004190] |
| Acey_2012.08.05_0010.g870 | 2.5688646986668e−05 | + | metalloendopeptidase activity |
| | | | [GO:0004222] |
| Acey_2012.08.05_0154.g3014 | 3.20320683408809e−05 | | cysteine-type endopeptidase activity |
| | | | [GO:0004197] |
| Acey_2012.08.05_0781.g2301 | 6.19309251932648e−05 | | aspartic-type endopeptidase activity |
| | | | [GO:0004190] |
| Acey_2012.08.05_0103.g3579 | 6.80585558556827e−05 | | serine-type endopeptidase activity |
| | | | [GO:0004252] |
| Acey_2012.08.05_0048.g1548 | 9.75483227451861e−05 | | serine-type peptidase activity |
| | | | [GO:0008236] |
| Acey_2012.08.05_0641.g1028 | 0.000108178832755462 | | metalloendopeptidase activity |
| | | | [GO:0004222] |
| Acey_2012.08.05_0154.g3007 | 0.000110274586983036 | + | cysteine-type endopeptidase activity |
| | | | [GO:0004197] |
| Acey_2012.08.05_0273.g989 | 0.00012602789625187 | | metalloendopeptidase activity |
| | | | [GO:0004222] |
| Acey_2012.08.05_0195.g1489 | 0.000132057626602926 | + | aspartic-type endopeptidase activity |
| | | | [GO:0004190] |
| Acey_2012.08.05_0195.g1490 | 0.000147000668090609 | | aspartic-type endopeptidase activity |
| | | | [GO:0004190] |
| Acey_2012.08.05_0288.g1485 | 0.000151166165541015 | | serine-type peptidase activity |
| | | | [GO:0008236] |
| Acey_2012.08.05_0028.g1777 | 0.000192264887352063 | | cysteine-type endopeptidase activity |
| | | | [GO:0004197]; cysteine-type |
| | | | endopeptidase inhibitor activity |
| | | | [GO:0004869] |
| Acey_2012.08.05_0220.g2503 | 0.00027036875982883 | + | cysteine-type endopeptidase activity |
| | | | [GO:0004197] |
| Acey_2012.08.05_0247.g51 | 0.000344982367250545 | | cysteine-type endopeptidase activity |
| | | | [GO:0004197] |
| Acey_2012.08.05_0038.g3641 | 0.000399488164035572 | | metalloendopeptidase activity |
| | | | [GO:0004222] |
| Acey_2012.08.05_0038.g3645 | 0.000497062972299171 | | metalloendopeptidase activity |
| | | | [GO:0004222] |
| Acey_2012.08.05_0010.g888 | 0.000645621348658581 | | metalloendopeptidase activity |
| | | | [GO:0004222] |
| Acey_2012.08.05_0144.g2437 | 0.000673566965394102 | | cysteine-type endopeptidase activity |
| | | | [GO:0004197] |
| Acey_2012.08.05_0195.g1492 | 0.000704720293510121 | | aspartic-type endopeptidase activity |
| | | | [GO:0004190] |
| Acey_2012.08.05_0001.g224 | 0.000772064015632964 | + | metalloendopeptidase activity |
| | | | [GO:0004222] |
| Acey_2012.08.05_0173.g421 | 0.000782307702805552 | | serine-type peptidase activity |
| | | | [GO:0008236] |
| Acey_2012.08.05_0195.g1494 | 0.000839763966008027 | + | aspartic-type endopeptidase activity |
| | | | [GO:0004190] |
| Acey_2012.08.05_0619.g718 | 0.000863173922670397 | + | cysteine-type endopeptidase activity |
| | | | [GO:0004197] |
| Acey_2012.08.05_0230.g2983 | 0.000891216697808933 | | metalloendopeptidase activity |
| | | | [GO:0004222] |
| Acey_2012.08.05_0018.g3533 | 0.000921383010005993 | | cysteine-type endopeptidase activity |
| | | | [GO:0004197] |

*Significance of upregulation from L3i to 24.PI was computed by edgeR; smaller q-values denote more pronounced upregulation.
+ Predicted by Phobius to be secreted

TABLE 14
Genes in A. ceylanicum that are significantly upregulated in
early infection (from L3i to 24PI) which encode protease inhibitors,
and which lack obvious homology to mammalian proteins.

| Gene | q-value | Secreted |
|---|---|---|
| Acey_2012.08.05_0056.g2712 | 9.77697175251355e−08 | + |
| Acey_2012.08.05_0833.g2587 | 8.38157936771835e−07 | + |
| Acey_2012.08.05_0010.g1216 | 5.99507919103354e−06 | + |
| Acey_2012.08.05_0016.g3109 | 0.000206283167326564 | |
| Acey_2012.08.05_0005.g2371 | 0.000386869470360868 | + |
| Acey_2012.08.05_0016.g3111 | 0.000486601741317141 | + |
| Acey_2012.08.05_0016.g3121 | 0.000645621348658581 | |

The significance of upregulation from L3i to 24.PI was computed by edgeR; smaller q-values denote more pronounced upregulation. Genes whose products are predicted by Phobius to be secreted are noted with ‘+’.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A nucleic acid comprising:
a nucleotide sequence encoding an amino acid sequence comprising at least 10 consecutive amino acids encoded by an open reading frame in any one of SEQ ID NOS:1-540; and
a promoter operably linked to the nucleotide sequence, wherein the promoter is not a hookworm promoter.

2. The nucleic acid of claim 1, wherein the amino acid sequence has at least about 95% sequence homology with an amino acid sequence comprising at least 20 consecutive amino acids encoded by an open reading frame in any one of SEQ ID NOS:1-540.

3. The nucleic acid of claim 2, wherein the amino acid sequence comprises an amino acid sequence having at least 95% sequence homology with an amino acid sequence encoded by any one of SEQ ID NOS:1-540.

4. The nucleic acid of claim 1, wherein the promoter can drive the transcription of the nucleotide sequence in a bacterium, yeast, fungal cell, plant cell, insect cell, or mammalian cell.

5. The nucleic acid of claim 4, wherein the promoter can drive transcription of the nucleotide sequence in Escherichia coli, Bacillus subtilis, Pseudomonas fluorescens, Leishmania tarentolae, Saccharomyces cerevisiae, Pichia Pastoris, Nicotiana, Drosophila melanogaster, Spodoptera frugiperda, Trichoplusia ni, Gallus gallus, Mus musculus, Sus scrofa, Ovis aries, Capra aegagrus, Bos taurus, Sf9 cells, Sf21 cells, Schneider 2 cells, Schneider 3 cells, High Five cells, NS0 cells, Chinese Hamster Ovary (“CHO”) cells, Baby Hamster Kidney cells, COS cells, Vero cells, HeLa cells, or HEK 293 cells.

6. The nucleic acid of claim 5, wherein the promoter can drive transcription of the nucleotide sequence in Escherichia coli, Saccharomyces cerevisiae, or CHO cells.

7. A method for transfecting a cell, comprising transfecting a cell with the nucleic acid claim 1.

8. The method of claim 7, wherein the cell is selected from Escherichia coli, Bacillus subtilis, Pseudomonas fluorescens, Leishmania tarentolae, Saccharomyces cerevisiae, Pichia Pastoris, Nicotiana, Drosophila melanogaster, Spodoptera frugiperda, Trichoplusia ni, Gallus gallus, Mus musculus, Sus scrofa, Ovis aries, Capra aegagrus, Bos taurus, Sf9 cells, Sf21 cells, Schneider 2 cells, Schneider 3 cells, High Five cells, NS0 cells, Chinese Hamster Ovary (“CHO”) cells, Baby Hamster Kidney cells, COS cells, Vero cells, HeLa cells, and HEK 293 cells.

9. A cell comprising the nucleic acid of claim 1.

10. The cell of claim 9, wherein the cell is selected from Escherichia coli, Bacillus subtilis, Pseudomonas fluorescens, Leishmania tarentolae, Saccharomyces cerevisiae, Pichia Pastoris, Nicotiana, Drosophila melanogaster, Spodoptera frugiperda, Trichoplusia ni, Gallus gallus, Mus musculus, Sus scrofa, Ovis aries, Capra aegagrus, Bos taurus, Sf9 cells, Sf21 cells, Schneider 2 cells, Schneider 3 cells, High Five cells, NS0 cells, Chinese Hamster Ovary (“CHO”) cells, Baby Hamster Kidney cells, COS cells, Vero cells, HeLa cells, and HEK 293 cells.

11. A method for producing an antigen, comprising incubating the cell of claim 9 under conditions sufficient to express the nucleotide sequence, thereby producing the antigen.

12. A method for preventing or treating a hookworm infection in a subject, comprising administering to the subject a composition comprising either an antigen or a nucleic acid encoding the antigen, wherein the antigen comprises an amino acid sequence comprising at least 10 consecutive amino acids encoded by an open reading frame in any one of SEQ ID NOS:1-540.

13. The method of claim 12, wherein the amino acid sequence has at least about 95% sequence homology with an amino acid sequence comprising at least 20 consecutive amino acids encoded by an open reading frame in any one of SEQ ID NOS:1-540.

14. The method of claim 13, wherein the amino acid sequence comprises an amino acid sequence having at least 95% sequence homology with an amino acid sequence encoded by any one of SEQ ID NOS:1-540.

15. The method of claim 12, wherein the subject is selected from murines, felines, canines, ovines, porcines, bovines, equines, and primates.

16. The method of claim 15, wherein the subject is selected from Felis catus, Canis lupus familiaris, and Homo sapiens.

17. A method for modulating an immune response in a subject, comprising administering to the subject a composition comprising either:
a peptide or protein; or
a nucleic acid encoding the peptide or protein;
wherein the peptide or protein comprises an amino acid sequence comprising at least 10 consecutive amino acids encoded by an open reading frame in any one of SEQ ID NOS:1-203 and SEQ ID NOS:405-540.

18. The method of claim 17, wherein administering the composition to the subject decreases an immune response in the subject.

19. The method of claim 17, wherein the subject is selected from murines, felines, canines, ovines, porcines, bovines, equines, and primates.

20. The method of claim 19, wherein the subject is selected from Homo sapiens and Mus musculus.

21. A peptide or protein comprising an amino acid sequence comprising at least 10 consecutive amino acids encoded by an open reading frame in any one of SEQ ID NOS:1-540.

22. The peptide or protein of claim 21, wherein the amino acid sequence has at least about 95% sequence homology with an amino acid sequence comprising at least 20 consecutive amino acids encoded by an open reading frame in any one of SEQ ID NOS:1-540.

23. The peptide or protein of claim 22, wherein the amino acid sequence comprises an amino acid sequence having at least 95% sequence homology with an amino acid sequence encoded by any one of SEQ ID NOS:1-540.

24. A sterile, injectable pharmaceutical composition, comprising the peptide or protein of claim 21.
